SEQUENCE LISTING (10 sequences)

SEQ ID NO: 1 - protein, 470 aa, Homo sapiens
GKLPLKAKMGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 2 - DNA, 1730 bp, Homo sapiens
ggaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaaaaaaaaaaaaaaaaaaaaaaaa

SEQ ID NO: 3 - RNA, 19 nt, Artificial
  synthetic oligonucleotide siRNA anti-eEF1A1 sens
uggugacaacaugcuggag

SEQ ID NO: 4 - RNA, 19 nt, Artificial
  synthetic oligonucleotide siRNA anti-eEF1A1 antisens
cuccagcauguugucacca

SEQ ID NO: 5 - protein, 331 aa, Homo sapiens
APASPMLGNASNPNKADIPERKKSSTVPSSNTASGGMTRRNTYVCSERTTADRHSVIQNGKENSLTEMFAYAASPASVCTSTCRLRHQKSMSMSASGHPKMMLPPIDSEGDNFKAITIPDQRTPVASTHSISSAATPDRIRFPRGTASRSTFHGQPRERRTATYNGPPASPSLSHEATPLSQTRSRGSTNLFSKLTSKLTRRLPTEYERNGRYEGSSRNVSAEQKDENKEAKPRSLRFTWSMKTTSSMDPGDMMREIRKVLDANNCDYEQRERFLLFCVHGDGHAENLVQWEMEVCKLPRLSLNGVRFKRISGTSIAFKNIASKIANELKL

SEQ ID NO: 6 - DNA, 1122 bp, Homo sapiens
gctccagccagtcccatgcttgggaatgcaagtaatcctaataaggcggatattcctgaacgcaagaaaagctccactgtccctagtagtaacacagcatctggtggaatgacacgacgaaatacttatgtttgcagtgagagaactacagctgatagacactcagtgattcagaatggcaaagaaaacagcctgacagaaatgttcgcttacgcagctagccctgcctcagtttgtacatctacctgtcggctgagacatcagaagtcgatgtccatgtcagcctctgggcaccccaagatgatgttacctccaatagacagtgaaggagataacttcaaggctatcactattcctgatcagagaactccagttgcttcaacacacagtatcagtagtgcagccaccccagatcgaatccgcttcccaagaggcactgccagtcgtagcactttccacggccagccccgggaacggcgaaccgcaacatataatggccctcctgcctctcccagcctgtcccatgaagccacaccattgtcccagactcgaagccgaggctccactaatctctttagtaaattaacttcaaaactcacaaggaggcttccaactgaatatgagaggaacgggagatatgagggctcaagtcgcaatgtatctgctgagcaaaaagatgaaaacaaagaagcaaagcctcgatccctacgcttcacctggagcatgaaaaccactagttcaatggatcccggggacatgatgcgggaaatccgcaaagtgttggacgccaataactgcgactatgagcagagggagcgcttcttgctcttctgcgtccacggagatgggcacgcggagaacctcgtgcagtgggaaatggaagtgtgcaagctgccaagactgtctctgaacggggtccggtttaagcggatatcggggacatccatagccttcaaaaatattgcttccaaaattgccaatgagctaaagctgtaacccagtgattatgatgtaaattaagtagcaattaaagtgttttcctgaacactgaaaaaaaaaaaaaaaaaaaaaaaaaaaaaaaaaaaaaaaaaaaaaaaaaaaaaaaaaaaaaaaaaaaaaaaa

SEQ ID NO: 7 - RNA, 19 nt, Artificial
  synthetic oligonucleotide siRNA anti-MARK3 sens
acagcacuauuccugauca

SEQ ID NO: 8 - RNA, 19 nt, Artificial
  synthetic oligonucleotide siRNA anti-MARK3 antisens
ugaucaggaauagugcugu

SEQ ID NO: 9 - RNA, 19 nt, Artificial
  synthetic oligonucleotide siRNA anti-2C10 sens
ccuccaauagacagugaag

SEQ ID NO: 10 - RNA, 19 nt, Artificial
  synthetic oligonucleotide siRNA anti-2C10 antisens
cuucacugucuauuggagg

(19) United States
(12) Patent Application Publication
(10) Pub. No.: US 2009/0175871 A1
(43) Pub. Date: Jul. 9, 2009

(54) METHOD FOR DEMONSTRATING PRESENCE OR ABSENCE OF MARKERS ASSOCIATED WITH THE PRESENCE AND/OR THE CHEMOSENSITIVITY OF TUMORS

(75) Inventors: Laurent Pelletier, Echirolles (FR); Sandie Marand, Grenoble (FR); Jean-Paul Issartel, Saint Egreve (FR); Francois Berger, Meylan (FR); Rejane Beugnot, Meylan (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Institut National de la Sante et de la Recherche Medicale (INSERM), Paris (FR)

(21) Appl. No.: 12/094,880

(22) PCT Filed: Nov. 27, 2006
(86) PCT No.: PCT/EP2006/068943

§ 371 Date: May 23, 2008

(30) Foreign Application Priority Data
Nov. 25, 2005 (FR) 0511954
Nov. 25, 2005 (FR) 0511958

(51) Int. Cl. A61K 39/395; C12Q 1/02; G01N 33/68; G01N 33/536; C12Q 1/48; C07K 16/18; C12N 5/06; C07H 21/02; C12N 15/63; C12N 9/12; C07H 21/00; C12N 5/10; C12P 21/02; A61K 31/7105; A61P 35/00

(52) U.S. Cl. 424/139.1; 435/29; 436/86; 436/536; 435/15; 530/387.9; 435/375; 536/24.5; 435/320.1; 435/194; 536/23.2; 435/325; 435/69.1; 514/44

(57) ABSTRACT

A method for detecting presence or absence of a tumor in a mammal and/or its sensitivity to chemotherapies, including, on a biological sample from said mammal, detecting and/or quantifying: presence of an eEF1A1 protein, and/or presence of antibodies directed against an eEF1A1 protein or a fragment including at least one epitope of eEF1a1 protein, and/or presence of a MARK3 protein, and/or presence of antibodies directed against a MARK3 protein or a fragment comprising at least one epitope of the MARK3 protein.

### RELATED APPLICATION

This is a §371 of International Application No. PCT/EP2006/068943, with an international filing date of Nov. 27, 2006 (WO 2007/060240 A2, published May 31, 2007), which is based on French Patent Application Nos. 05/11954, filed Nov. 25, 2005, and 05/11958, filed Nov. 25, 2005.

### TECHNICAL FIELD

This disclosure relates novel methods with which to evidence the presence of absence of markers associated with tumors and their sensitivity to chemotherapies. The disclosure also relates diagnostic kits comprising means enabling the methods to be implemented, and relates the use of compounds inhibiting the activity or expression of the markers to inhibit the growth of tumor cells.

### BACKGROUND

In the area of pathologies, cancers occupy a predominant position in terms of prevalence, incidence and mortality. Cancer-forming phenomena are related to complex cell disorders, not well known, which may affect all the organs. The means for screening and fighting cancers remain imperfect.

There is a large variety of tumor phenomena which may occur at any age of an individual and affect most functional areas of a human body. In particular, the central nervous system (CNS), a complex organ consisting of numerous different cell types, is no exception to these morbid and most diversified pathological phenomena. Indeed there are numerous types of solid brain tumors. These tumors correspond to the development of oncological phenomena affecting the constituent cells of the CNS (neuronal cells, glial cells.). Also there exist other localized tumors in the CNS which are the result of metastases derived from tumors of other organs. CNS tumors are characterized by a certain number of anatomical, biological and clinical parameters. At the current time, careful analysis of these parameters predominantly determines the strategy of therapeutic action taken by the clinician. The specific phenotype of a tumor is an element which, still most imperfectly, allows the prognostic evaluation of a patient's chances of survival.

Amongst the investigations which can be used to classify brain tumors, mention may be made of:
medical imaging (tomodensitometry and magnetic resonance imaging)
morphological and histological analysis performed on biopsies
biomolecular analysis: search for protein markers by immune-detection, cytogenetic analysis (e.g., detection of genetic macro-anomalies by probe hybridization).

The diagnosis of tumors and more particularly of CNS tumors is predominantly based on histological analysis conducted by an anatomo-pathologist. Unfortunately diagnostic discrepancies observed between experts in the area are enormous (up to 64% disagreement depending on tumors). Worse still, similar discrepancies can be noted when the interpretations of identical samples are given to the same person at a few weeks' interval (Mittler et al, 1996; Bruner et al, 1997; Coons et al, 1997). This finding is worrying since it is known that diagnosis errors may lead to unnecessary radiotherapy and/or chemotherapy with heavy consequences for the patient.

As a supplement to histological analysis, there are only a few rare diagnosis tests based on molecular approaches. Cytological observations for example can be completed by the search for genetic anomalies and, in some laboratories, by the detection of certain protein markers using specific antibodies.

At the present time no routine tests exist based on the detection and quantification of the concentration of transcriptomic tumor markers. The few molecular tests currently available do not allow a non-ambiguous differentiation to be made between the different types of tumor cells, and especially do not allow correct prognosis of their sensitivity to cytotoxics with accuracy.

It is therefore important to be able to have new methods available allowing the easy, sensitive and early detection of the presence of tumors and their sensitivity to chemotherapies, to apply therapeutic strategies best adapted for the treatment of each patient.

### SUMMARY

We provide a method for detecting presence or absence of a tumor in a mammal and/or its sensitivity to chemotherapies, including, on a biological sample from the mammal, detecting and/or quantifying: presence of an eEF1A1 protein, and/or presence of antibodies directed against an eEF1A1 protein or a fragment including at least one epitope of the eEF1A1 protein, and/or presence of a MARK3 protein, and/or presence of antibodies directed against a MARK3 protein or a fragment including at least one epitope of the MARK3 protein.

We also provide antibodies directed against an eEF1A1 protein or a fragment containing at least one epitope of the eEF1A1 protein that bind specifically to the eEF1A1 protein or to at least one epitope of the eEF1A1 protein.

We further provide antibodies directed against a MARK3 protein or a fragment containing at least one epitope of the MARK3 protein that bind specifically to the MARK3 protein or to at least one epitope of the MARK3 protein.

We still further provide a method of inhibiting growth of tumor cells in vitro including inhibiting activity of an eEF1 A1 protein and/or of a MARK3 protein by an antibody or an interfering RNA which inhibits expression of a gene encoding the eEF1A1 protein and/or the MARK3 protein, respectively.

We also further provide interfering RNA that inhibits in vitro and/or in vivo expression of a gene encoding an eEF1A1 protein or a gene encoding a MARK3 protein.

We further yet provide a variant of a MARK3 protein comprising the protein sequence of SEQ ID NO: 5.

### BRIEF DESCRIPTION OF THE DRAWINGS

FIG. 1: Western-blot conducted with a cystic fluid tested to evidence reactions vis-à-vis proteins of tumor origin. Three different samples were used to generate protein imprints, after electrophoresis, for the immune-detection reaction:
Lane M: protein extract from membranes of glioblastoma cells;
Lane C: cytoplasmic fraction of glioblastoma cells;
Lane T: total extract of glioblastoma cells.

FIG. 2: Sequence of the cDNA of clone IG5 (A) and of the expressed protein (B).

FIG. 3: Alignment of the protein sequence expressed by the IG5 clone with the sequence of the C ter end of the isoform 4 protein of eEF1A1.

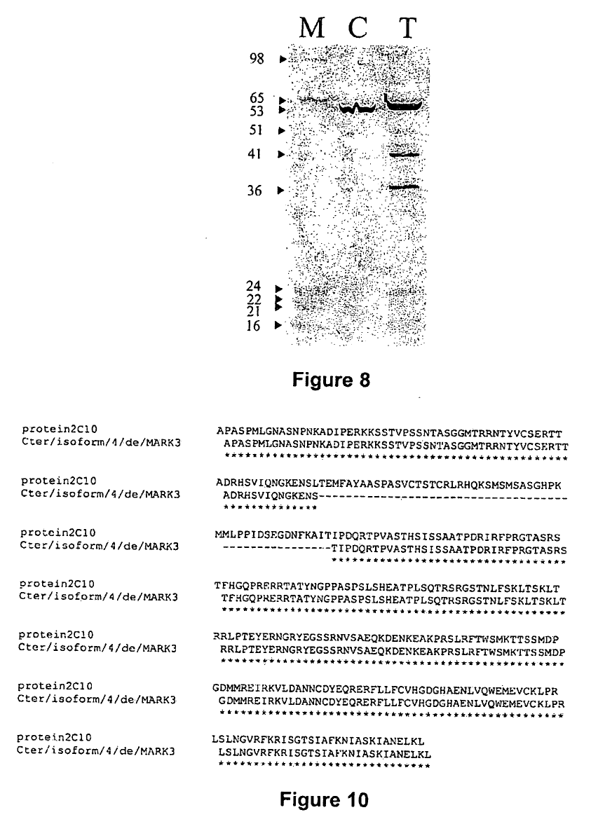

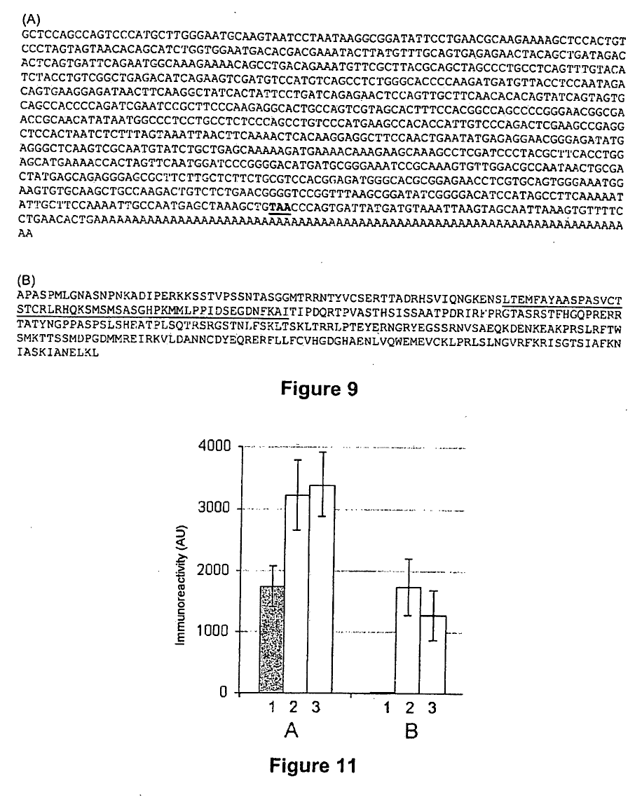

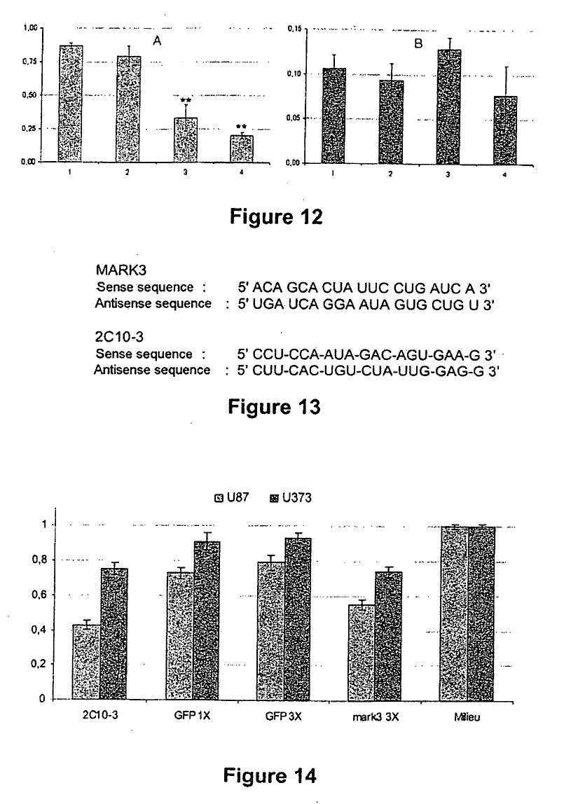

FIG. 4: Measurement of the intensity of immunoreactions detected in the sera of different groups of individuals vis-à-vis the antigen protein encoded by the 1G5 clone. The groups of individuals consist of:
Group 1: normal individuals, controls;
Group 2: patients with gliomas responding positively to chemotherapy;
Group 3: patients with gliomas not responding to chemotherapy.
The immunoreactivity histograms correspond to the signals recorded in, Western-blots (expressed in arbitrary units, AU) with the following antigens:
   A: total protein expressed by the 1G5 clone (corresponding to the domain of the isoform 4 variant of eEF1A1 described in Example 3, FIG. 3);
   B: fragmentation product of 12 kDa from the domain of the isoform 4 variant of eEF1A1 described in Example 3.

FIG. 5: Measurement of the cytotoxicity of purified immunoglobulins of cystic fluids or sera on culture tumor cells.

FIG. 6: Sequences of the siRNA tested in vitro.

FIG. 7: Graph of transfection tests of cell lines U138, GHD, U373 and U87.

FIG. 8: Western-blot performed with a cystic fluid to evidence reactions vis-à-vis proteins of tumor origin. Three different samples were used to generate protein imprints, after electrophoresis, for the immune-detection reaction:
Lane M: protein extract from membranes of glioblastoma cells;
Lane C: cytoplasmic fraction of glioblastoma cells;
Lane T: total extract of glioblastoma cells.

FIG. 9: cDNA sequence of the 2C10 clone (A) and of the expressed protein (B).

FIG. 10: Alignment of the protein sequence expressed by the 2C10 clone with the sequence of the C ter end of the isoform 4 protein of MARK3.

FIG. 11: Measurement of the intensity of immunoreactions detected in the sera of different groups of individuals vis-à-vis the antigen protein encoded by clone 2C10. The groups of individuals consist of:
Group 1: normal individuals, controls;
Group 2: patients with gliomas responding positively to chemotherapy;
Group 3: patients with gliomas not responding to chemotherapy.
The immunoreactivity histograms correspond to the signals recorded in Western-blots (expressed in arbitrary units, AU) with the following antigens:
   A: total protein expressed by the 2C10 clone (corresponding to the domain of the isoform 4 variant of MARK3 described in Example 9, FIG. 10);
   B: fragmentation product of 11 kDa from the domain of the isoform 4 variant of MARK3 described in Example 9.

FIG. 12: Measurement of the cytotoxicity of the purified immunoglobulins of cystic fluids or sera on culture tumor cells.

FIG. 13: Sequences of the siRNAs directed against MARK3 and against its isoform 4 tested in vitro.

FIG. 14: Measurement of the cytotoxicity of the siRNAs directed against MARK3 and against its isoform 4 on culture tumor cells.

### DETAILED DESCRIPTION

We therefore provide novel diagnostic methods to detect the presence or absence of a tumor and its sensitivity to chemotherapies in a mammal, in particular in man, by detection and/or quantification of the presence of a novel biological marker in a biological sample previously taken from the mammal: protein eEF1A1 (elongation factor of protein synthesis, Swiss-Prot reference: http://www.expasy.org/uniprot/P68104, the subject matter of which is incorporated by reference.

The absence of an eEF1A1 protein, or of antibodies directed against an eEF1A1 protein, or a fragment comprising at least one epitope of the protein, or a low concentration compared with concentrations observed in healthy individuals or patients suffering from cancers sensitive to chemotherapies, is characteristic of the presence of a tumor most likely to be resistant to usual chemotherapies (chemoresistant).

Conversely, a level of eFLAI protein, or of antibodies directed against an eEF1A1 protein, or a fragment comprising at least one epitope of the protein, that is comparable to the level observed in healthy persons is characteristic of a tumor most likely to be sensitive to usual chemotherapies (chemosensitive).

We also provide novel diagnostic methods to detect the presence or absence of a tumor in a mammal, in particular man, by detection and/or quantification of the presence of a novel biological marker in a biological sample previously taken from the mammal: a MARK3 protein (Genbank accession number AF 387637; Sun T.-Q et al “PAR-1 is a Dishevelled-associated kinase and a positive regulator of Wnt signalling” Nat. Cell. Biol. 2001, 3, 628-636), the subject matter of which is incorporated by reference. The presence of a MARK3 protein or of antibodies directed against a MARK3 protein, or against a fragment comprising at least one epitope of the protein is characteristic of the presence of a tumor.

We therefore also provide methods which can be used to detect the presence or absence, in a mammal, of a tumor and/or its sensitivity to chemotherapies, the method comprising a step to detect and/or quantify in a biological sample previously taken from the mammal:
presence of an eEF1A1 protein, and/or
presence of antibodies directed against an eEF1A1 protein or a fragment comprising at least one epitope of this protein, and/or
presence of a MARK3 protein, and/or
presence of antibodies directed against a MARK3 protein or a fragment comprising at least one epitope of this protein.

By eEF1 A1 protein is meant a protein which comprises a protein sequence of the reference eEF1A1 protein (Swiss-Prot: http://www.expasy.org/uniprot/P68104), its isoforms, its variants and its biologically active fragments.

By MARK3 protein is meant a protein which comprises a protein sequence of the reference MARK3 protein, described in Genbank under accession no AF387637 (Sun T.-Q et al “PA1 is a Dishevelled-associated kinase and a positive regulator of Wnt signaling,” Nat. Cell Biol, 628-636), its isoforms, its variants and its biologically active fragment.

The isoforms fragments or biologically active variants are recognized by anti-eEF1A1 antibodies or anti-MARK3 antibodies, respectively.

By variants is meant proteins which advantageously have at least 75% identity with the reference eEF1A1 protein or with the reference MARK3 protein, respectively, more preferably at least 80%, and further preferably at least 85% identity or still further preferably at least 95% identity.

The methods of sequence alignment and identity calculation are well known to persons skilled in the art and are directly accessible on the Internet. Particular mention is made of the BLAST program which can be used from the site http://www.ncbi.nlm.rih.gov/BLAST/ with the default parameters indicated on this site. Advantageous use may also be made of advanced BlastP search, by fine-tuning search with a (PHI-BLAST) pattern. For sequence alignment, it is possible to use the programmes CLUSTALW (http://www.ebi.ac.uk/clustalw/) or MULTALIN (http://prodes.toulouse.inra.fr/multalin/), with the default parameters indicated on these sites.

The differences between the variants of eEF1A1 or MARK3 and their respective reference sequence may be due to deletions of at least one amino acid, and when several amino acids are deleted they may be contiguous or separated on the reference sequence. The differences may also be due to mutations, at least one amino acid being replaced by a different amino acid in the reference sequence. The differences may also be due to the addition of at least one amino acid in the reference sequence. When several amino acids are added to the reference sequence, they may be contiguous, i.e., forming a protein fragment of at least 2 amino acids, or distributed on the sequence, or both.

The eEF1A1 variant may comprise at least one protein fragment inserted in the reference eEF1A1 sequence. This fragment may comprise up to 100 amino acids, generally between 10 and 60 amino acids.

The eEF1A1 protein may comprise the protein sequence given under SEQ ID NO: 1.

The MARK3 variant may comprise at least one protein fragment inserted in the reference MARK3 sequence. This fragment may comprise up to 100 amino acids, generally between 10 and 60 amino acids.

The MARK3 protein may be a variant comprising the protein sequence given under SEQ ID NO: 5.

Advantageously the biological sample previously taken is chosen from among a sample of blood serum, lymph, cystic fluid, or tissue homogenate, preferably a sample of blood serum.

Evidently, depending on the method of detection and/or quantification used, the biological sample may undergo treatment prior to its analysis, e.g., grinding and/or dissolution. The treatments are well known to those skilled in the art with respect to detection methods.

The detection and/or quantification means of the presence of a protein or antibody in a biological sample are well known to those skilled in the art, and in particular are described below and in the examples.

The methods described below are given by way of indication and in no manner amount to an exhaustive list of the technical approaches which can be used. Persons skilled in the art, according to technical developments and optimization, will know at any time which methods are best adapted for the conducting of analyses to implement the disclosure while advantageously substituting the measurement and/or immuno-reaction methods described herein.

The methods are given below divided into groups for simplification:
Tests involving capture on a support;
Detection tests based on macroscopic evidencing of immune complexes;
Tests based on isotopic marking and radio-immune assays (RIAs);
Analyses based on immunohistochemical techniques;
Tests based on fluorescence transfer methods (Fluorescence Resonance Energy Transfer—FRET) or energy transfer methods using luminescence (Bioluminescence Resonance Energy Transfer—BRET).

### Tests Entailing Capture on a Support

In the methods described below, mention is made of the use of antigens corresponding to an eEF1A1 protein, a MARK3 protein or to a fragment of one of these proteins comprising at least one epitope recognized by the anti-EF1A1 antibodies or anti-MARK3 antibodies, respectively. It is therefore to be appreciated that the tests can also be conducted with only fragments of these proteins-antigens (natural or synthetic or chimerical peptides . . . ) or compounds and molecules of haptene type able to react selectively with the anti-eEF1A1 or anti-MARK3 antibodies whose presence in the sera it is sought to detect.

The Western-blot and ELISA methods described below are two methods from among those most frequently used for these types of test. Other methods to detect antigen-antibody reactions can be used using various modes of interactions or adsorptions of components with supports, reaction wells or microdetection systems. These tests very often combine high performance levels, related to the high sensitivities of the methods, with relative ease of sample handling, test robustness and high speed treatment capacities of numerous samples simultaneously.

### a) Method Based on Western-Blot (Towbin et al, 1979; Burnette, 1981)

To carry out this method, an extract containing at least one antigen is subjected to electrophoretic migration on polyacrylamide gel in a denaturing medium. This technique is well known in one of its variants under the abbreviation “SPAGE: SDS PolyAcrylamide Gel Electrophoresis” (SDS: Sodium Doceyl Sulfate). Electrophoresis migration allows proteins to be separated in relation to their respective molecular weights (Laemmli, 1970). After this separation, and following conventional protocols known to persons skilled in the art, imprints of proteins are made on a membrane of nylon type and are placed in incubation with the sera to be analyzed. Western-blot detection protocols, most conventional, can reveal whether the antibodies which may have been present in the sera have fixed themselves onto the antigens fixed on the imprint. The protocols use radioactive, fluorescent or luminescent techniques to demonstrate the presence of the antibodies. The analysis can be conducted so as to access a quantitative estimation of the antibody level in the initial sample that is sufficiently precise to give a diagnosis of clinical interest. This type of test can be conducted using Western blot equipment distributed by Immunetics (Boston, Mass., USA; http://www.immunetics.com).

### b) ELISA Method (and it Derivatives)

One henceforth conventional method called ELISA (Enzyme Linked Immuno Sorbent Assays) (Engvall and Perlmann, 1971, 1972; Engvall et al, 1.971) consists of causing the creation of antigen-antibody complexes (immune complexes) in immobilized form on the walls of wells of multi-well assay plates in plastic material. This type of method exists in a multitude of variants depending on whether the protocols are based on the primary immobilization of antibodies or antigens at the bottom of the wells, and depending on the development methods used (direct or indirect ELISA). By way of example, it can briefly be mentioned that this type of test can be carried out according to the description given below for the detection of antibodies directed against the antigen. The wells of the ELISA assay plate are filled individually and independently with increasing dilutions of antigen. The proteins fix by adsorption to the bottom of the wells. After washing the wells, the proteins adhering to the walls of the wells are placed in contact with the antibodies to be detected and present in the sera. Therefore, if they are present in the sera, the antibodies are immobilized at the bottom of the well by fixing to the antigen proteins adsorbed on the wall of the well. A detection step (based for example on a simple colorimetric test) to detect the presence of antibodies at the bottom of the wells therefore gives an indication of the presence of these antibodies in the initial serum sample. The conducting of assays on dilutions of the sample allows a quantitative estimation of the level of antibodies in the biological sample. ELISA test analyses can be conducted on apparatus such as the VIDAS system distributed by Bio-Merieux (http://www.biomerieux.com).

### c) Methods Based on Protein Micro-Arrays

According to one approach to immobilize immune complexes, the micro-array technique is based on more or less extensive miniaturization, automation and parallelizing of the number of tests. For these tests it is necessary to create micro-arrays of proteins consisting of generally planar solid surfaces (glass slides, silica fragments, bottom of multi-well plates in plastic material . . . ) containing antigens fixed using various chemical methods onto the support, each antigen being deposited on a small surface of the support representing a surface area of a few square microns. For example, the antigens are immobilized on the support by depositing microdroplets of a suspension of the antigens on these supports (Peluso et al, 2003, Kusnezow and Hoheisel, 2003). After incubation with the samples to be analyzed, the formed immune complexes can be detected by various technical means, the most frequent being based on the detection of fluorescence signals, or a method using surface plasmon resonance (Vikinge et al, 1998; Kusnezow and Hoheisel, 2003).

Tests based on micro-arrays with fluorescence detection can be envisaged using protocols which even allow direct analysis on a sample of whole blood. The system such as the one proposed by Unedik (http://www.unedik.com) comprising micro-array and reader allows the analysis of several markers simultaneously with quantification of signal intensities.

Surface plasmon resonance is based on a well-known experimental physical principle in which the planar surface of a gold film reflects an incident light beam in a direction predicted by laws of conventional optics. Nonetheless, for a very small part of the reflected light beam and at a certain incidence, a significant reduction is found in the number of reflected photons. The angle of incidence of the reflection area of the least luminous zone depends on the quantity of matter fixed onto the face of the gold film opposite the face irradiated by the incident light beam. Any interaction of antibody, antigen or any formation of immune complex on the opposite face of a detector based on plasmon resonance and irradiated at a certain incidence by a light ray therefore causes a substantial change in the angle of reflection of the least luminous part of the reflected light beam. The detection of this deflection of the angle of reflection is used to evidence and quantify the fixing of components on the detector.

This latter detection method forms the base of a technique to analyze interactions between antibodies and proteins, and can be used to measure the kinetic parameters of interactions between antigens and antibodies, and to deduce therefrom the quantities of antibodies present in a sample (Fagerstam et al, 1990; Szabo et al, 1995). This technique is developed in the form of automated measuring apparatus of which one example is known under the name BIAcore (BIAcore AB, Uppsala, Sweden: http://www.biacore.com).

### d) Method Based on Mass Spectrometry

The detection of antibodies can be conducted using mass spectrometry for example called SELDI-TOF technology (Surface Enhanced Laser Desorption/Ionization—Time Of Fly technology) (Merchant and Weinberger, 2000; Weinberger et al, 2000). This embodiment is conducted using the technical platform distributed by Ciphergen (Ciphergen Biosystems, Inc. Fremont, Calif., USA; http://www.ciphergen.com). The detection of antibodies can be performed by previously immobilizing the antigens on the affinity supports which can be used for Ciphergen mass spectrometry or any other suitable mass spectrometry for this type of analysis. It is effectively possible to immobilize antigens by chemical grafting on silica, metal or polymer supports, and to use these supports to trap the antibodies present in a sample to be analyzed. The immune complexes thus formed and immobilized on the supports can then be analyzed by mass spectrometry. In this type of analysis the peak of immunoglobulins fixed on the antigens can be detected in the mass spectrum. The use of antigens grafted on the support provides for an extremely sensitive and specific assay method. Having regard firstly to the affinity of antibodies for the antigens, and secondly to the extensive detection sensitivity of mass spectrometry, even very minute quantities of antibodies present in a sample can be detected.

### Detection Tests Based on Macroscopic Evidencing of Immune Complexes (Latex Test, Immune-Detection Strips) (Singer et al, 1957; Hechemy et al, 1974)

In this type of detection system, the antigens of interest are chemically coupled to particle components of micrometric size such as polymer beads whether stained or not. Incubation, in a liquid or semi-liquid medium, of a fluid suspension of these beads coated with antigens together with the biological sample to be analyzed leads to the creation of immune complexes in which several polymer beads are aggregated. This aggregation translates as the formation of packets of beads whose size becomes macroscopically large to the point of becoming visible to an operator's “eye.” In one common, sophisticated variant of the system, the immune complexes are subjected to migration by capillarity on a chromatography support strip and developed through the creation of a colored band indicating the presence or absence of the detected antigen (test symbolized by immunodetection strips given wide, routine use for pregnancy tests for example). Latex tests are marketed by Bio-Merieux e.g., for microbiological diagnosis (www.biomerieux.com).

### Tests Based on Isotopic Marking and Radio-Immuno Assays (RIA) (Yalow and Berson, 1960; Booth et al, 1982).

In one of the variants of this assay, the immune complex is produced by adding a known quantity of antigen labelled with a radioactive isotope to the reaction medium, in addition to the serum sample. After selecting the immune complexes formed, the quantity of radioactivity detectable in the isolated fraction is proportional to the quantity of antibody present in a sample. Diagnostic kits using the RIA principle are distributed for various assays by Schering/Cis-Bio International (Gif/Yvette, France; www.cisbiointernational.fr). Immuno-assay can also be based on an assay principle which does not have recourse to radioactive tracers but uses fluorescence or luminescence markers.

### Analyses Based on Immunohistochemical Techniques (Kiernan, 1999)

One robust and relatively simple approach in its principle consists of an analysis of immuno-histological type on sections, smears or other preparations derived from biopsies. In this case they are tests conducted on an untreated sample (tumor sections consisting of more or less homogenous cells). The application of this technique is therefore more generally intended to evidence antigens in the preparation. The evidencing of formed immune complexes requires the detection of a signal generated by the use of radioactive tracers, or fluorescent reagents or calorimetric methods. Tumor cells containing the antigens will show a positive reaction with the selected reagent specific to the antigens. Conversely, non-tumor cells will not exhibit a signal or only a signal of significantly weaker intensity.

Tests based on cell sorting using fluorescence (so-called flow cytometry method or Fluorescent Activated Cell Sorting—FACS) (Hulett et al, 1969; Parks and Herzenberg, 1984): a reagent labelled with a fluorescent group can be used to label whole cells derived and isolated from biopsies, and to allow the sorting and quantification of cells that are positive for the presence of the antigens to be detected. Under this method the non-lysed cells, separated from freshly collected biopsy tissue, are contacted with the selective fluorescent reagent and the suspension is then analyzed using a cell sorter. The cell sorter detects the intensity of the fluorescent signal individually associated with each cell, and proceeds with counting the number of detected cells and optionally isolates the cells in specific reservoirs. Instruments designed for FACS analysis are distributed for example by Becton Dickinson (Franklin Lakes, N.J., USA; http://www.bd.com). The use of suitable optimized technical parameters (cell dilutions, optical parameters . . . ) allows selective sorting and counting of those cells containing the antigens, and ensures the feasibility of this method.

It is to be noted also that a flow cytometry approach applied to the analysis of sera could be conducted using spheres labelled with specific fluorescent compounds and containing antigens grafted on their surface. Immune complexes can then be evidenced, for example using the Bio-Plex technology by Bio-Rad (Hercules, Calif., USA; http://www.bio-rad.com); or the Cytometrix Bead Array technology by Becton Dickinson (http://bdeurope.com); or the LuminEx system by Miraibio (www://www/miraibio.com). Tests based on fluorescence transfer methods (Fluorescence Resonance Energy Transfer FRET; or Bioluminescence Resonance Energy Transfer—BRET)

For this type of analysis the assay, in its most attractive version, can be conducted directly in solution (homogenous phase assay) and does not require isolation or purification of one or other of the components of the immune complex. This method, in one of its variants, requires the use of two different antibodies directed against an antigen and labelled with suitable fluorescent groups. The two fluorescent groups are chosen so that their optical characteristics allow one thereof to be excited by the light ray used to measure fluorescence, then allow the transfer of the excitation energy to the second fluorescent group which ultimately emits a fluorescence radiation of specific wavelength. The transfer of fluorescence is only effective if the two molecules are maintained sufficiently close to one another. The two antibodies labelled with the two fluorescent groups are chosen so that they can fix themselves simultaneously on the antigens. The ternary immune complex formed (excitation fluorescent antibody—antigen—light emitting fluorescent antibody) therefore allows the two antibodies to be drawn close together and in this case only a fluorescence signal can be detected (at the emission wavelength of the emitting fluorescent group). This type of assay is rather more adapted, but not exclusively, to the assay of antigens. The intensity of the measured fluorescence signal is therefore directly proportional to the quantity of antigen present in the biological extract (Mathis, 1995; Szollosi et al, 1998; Blomberg et al, 1999; Ueda et al, 1999; Enomoto et al, 2000). Protein analysis using the fluorescence transfer method can be conducted on the Kryptor® apparatus made by the German company B.R.A.H.M.S. (www.brahms.de). Alternatively, the fluorescent compound excited by the excitation light ray in the FRET technique can be substituted by a bioluminescent system which is based on the activity of an enzyme (Xu et al, 1999).

The non-exhaustive list of techniques described above is just an example of the various techniques which persons skilled in the art are able to use for the analysis of biological samples when implementing the disclosure, to detect antibodies directed against the identified antigens, this analysis providing information of diagnostic or prognostic value.

The analyses can be conducted directly on non-treated samples or on treated samples, of which a non-exhaustive list includes lysates, extracts or subfractions derived from these samples.

The detection and assay of antibodies directed against the antigens described can be conducted using any products or derivatives derived from these antigens, and on their precursors provided they pay due heed to the specific recognition criteria for the antibodies to be detected.

As indicated previously, the assay of the antigens themselves or of their fragments or metabolically modified products, can itself form an analytical application of clinical interest.

The tests based on the use of SELDI-TOFF mass spectrometry require affinity supports for the specific capture of antigens (ProteinChip® arrays by Ciphergen). These supports are adapted in relation to the chosen capture mode: supports with chemical reactivity adapted for the fixing of antigens, or monoclonal or polyclonal antibodies recognizing all or part of the antigens or their peptides.

As will be obvious to those skilled in the art, for the needs of quantification and quality control (positive controls), the methods have recourse to suitable, standard proteins whether or not related to the antigens. Additionally, the immuno-detection tests require the use of components (which will be called reagents) able to interact with the antibodies of interest or the antigens and their derivatives. As such reagents mention may evidently be made of polyclonal or monoclonal antibodies and their immuno-reactive fragments, whether grafted or not on or with other components; particle elements able to interact with the antigens (phages or recombinant bacteria expressing polypeptide regions on their surface capable of interacting with haptenes or antigens (Gao et al, 1999; Knappik et al, 2000); or aptamers (chemical molecules of polynucleotide or even polypeptide type able to set up non-covalent interactions of strong affinity with the target molecules) (Ellington and Szostak, 1990; Tuerk and Gold, 1990). Specific reagents for the detection of immune complexes formed during tests can be chosen from among suitable conventional detection systems for this type of immuno-detection, such as Western-blot or ELISA (these reagents are secondary antibodies for example coupled to enzymatic systems allowing calorimetric reactions). The reagents are available by catalogue e.g., the catalogue by Sigma Aldrich, available on line (http://wvw.sigmaaldrich.com).

According to a first embodiment, the presence of the eEF1 A1 protein or MARK3 protein is detected and/or quantified using antibodies directed against the eEF1A1 protein or against the MARK3 protein, respectively, or at least one epitope of one of these proteins. The antibodies are advantageously chosen from among polyclonal antibodies or monoclonal antibodies.

The methods to identify and prepare the antibodies are known to those skilled in the art using usual techniques to prepare such antibodies. For reference, mention is made of the methods described in Immunobiology (5^{th} ed., Janeway, Charles A; Travers, Paul; Walport, Mark; Shlomchik, Mark. New York and London: Garland Publishing; c2001).

According to a second embodiment, the presence of antibodies directed against an eEF1A1 protein or against a MARK3 protein, respectively, or at least one epitope of one of these proteins, is detected and/or quantified by means of an antigen containing at least one epitope of an eEF1A1 protein or of a MARK3 protein, respectively.

According to one particular embodiment, the antigen comprises an EF1A1 protein such as defined above and below. Evidently the antigen may comprise mere fragments of an eEF1A1 protein on the understanding that the fragment comprises at least one epitope recognized by anti-eEF1A1 antibodies.

According to another embodiment, the antigen comprises a MARK3 protein such as defined above and below. Evidently the antigen may comprise mere fragments of a MARK3 protein, on the understanding that the fragment comprises at least one epitope recognized by anti-MARK3 antibodies.

An epitope is the smallest structural unit of an antigen recognized by an antibody, a structure present on the surface of the antigen molecule, capable of combining with a single anti-body molecule. From a structural viewpoint, epitopes can be of two types:
linear epitopes (short sequence of amino acids recognized by an antibody) having a size of around 8-10 amino acids, or
conformational epitopes, i.e., the antibodies recognize amino acids which lie close in space when the protein has its folded structure, but which are not located in immediate vicinity in the protein sequence.

It is to be noted that a given epitope can be recognized by several different antibodies generated under separate immunity reactions, related to different agents (viruses, bacteria, etc. . . . ). In this respect the level of recognition between the epitope and the different antibodies may vary from one epitope-antibody pair to another. For example, if the epitope is strongly recognized by the antibody, small antibody concentrations are sufficient to detect a recognition reaction between the epitope and the antibody. Conversely, if the epitope is weakly recognized by the antibody, strong antibody concentrations are required to detect a recognition reaction between the epitope and the antibody. Similarly, several separate epitopes may be recognized by one same antibody. But here again the levels of recognition from one epitope-antibody pair to another are generally variable.

The means to identify epitopes, antigen fragments, and to prepare antigens which can be used for a diagnosis method on biological samples are well known to those skilled in the art. Particular mention is made of a method consisting of synthesizing systematically those peptides capturing overlapping fragments of protein eEF1 A1 or protein MARK3, respectively, and then testing their capacity to stimulate an immune response.

Those skilled in the art will therefore be able to identify the epitope(s) best adapted to implement the method, through simple routine experimenting.

One fragment containing at least one epitope of the eEF1A1 protein is for example a fragment encompassing the N-terminal part of the eEF1A1 protein more particularly a fragment of 12 kDa encompassing this N-terminal part.

One fragment containing at least one epitope of the MARK3 protein is for example a fragment encompassing the N-terminal part of the MARK3 protein, more particularly a fragment of 11 kDA encompassing this N-terminal part.

Preferably the method comprises an additional step to compare the results obtained at the detection and/or quantification step with a reference value characteristic of the presence of a chemoresistant tumor and/or with a reference value characteristic of the presence of a chemosensitive tumor.

These reference values may differ depending on the means used for detection and/or quantification of eEF1 A1 or of anti-eEF1A1 antibodies. They may be obtained following usual methods in which the same analyses are conducted on samples derived from healthy individuals firstly and secondly from individuals known to be tumor carriers, a distinction being made in this second population between individuals known to have a chemosensitive tumor and those known to have a chemoresistant tumor.

According to one particular embodiment, the anti-MARK3 inhibitor specifically inhibits the variant of MARK3 whose protein sequence is given under SEQ ID NO: 5. By specific inhibition is meant that it inhibits the variant concerned, without substantially inhibiting the other variants of MARK3.

Evidently, the method may also comprise the detection and quantification of at least one other biological marker characteristic of the presence and/or invasiveness of a tumor and/or its chemosensitivity. As marker, one example of proliferation marker is the K167 protein antigen (SwissProt: http://www.expasy.org/uniprot; P46013) or phosphorylated vimentin whose absence is characteristic of an invasive tumor (PCT/EP2005/054598 filed on 15 Sep. 2005).

According to one particular embodiment, the method comprises a step, on a previously taken biological sample, to detect and/or quantify:
presence of an eEF1A1 protein, and/or
presence of antibodies directed against an eEF1A1 protein, or a fragment comprising at least one epitope of this protein, and
presence of a MARK3 protein, and/or
presence of antibodies directed against a MARK3 protein, or a fragment comprising at least one epitope of this protein.

The detection and/or quantification of the two markers can be conducted simultaneously, separately or at different times, on the same biological sample or on different samples.

We also provide diagnostic kits to implement the method such as defined above and below, the kit comprising means with which to detect and/or quantify, on a previously taken biological sample:
presence of an eEF1A1 protein, and/or
presence of antibodies directed against an eEF1A1 protein, or a fragment comprising at least one epitope of this protein, and/or
presence of a MARK3 protein, and/or
presence of antibodies directed against a MARK3 protein, or a fragment comprising at least one epitope of this protein.

The means are well known to persons skilled in the art, defined above, their form varying according to the chosen detection mode.

They firstly comprise an eEF1A1 or MARK3 antigen, respectively, defined previously, or an anti-eEF1A1 or anti-MARK3 antibody and reagents needed to carry out the diagnosis method.

The reagents are well known to those skilled in the art, and depend on the detection/quantification method used. They are notably described in the references cited above and in particular in the Sigma Aldrich catalogue available on line (http: /www.sigmaaldrich.com).

Advantageously the detection kit comprises a suitable support able to receive the biological sample, and the appropriate detection means. If the detection means is an antibody or an antigen, or their fragments, it may be bound to the support by any suitable means, e.g., by covalent bonding or adsorption on the support. The supports are well known to those skilled in the art, and are described in particular in the references given previously.

We also provide the antibodies directed against an eEF1A1 protein or against a MARK3 protein, or against a fragment comprising at least one epitope of one of these proteins, which bind specifically to an eEF1A1 protein or a MARK3 protein, respectively, or to at least one epitope of one of these proteins defined above and below. The disclosure also relates the antibodies for their therapeutic use.

In addition to eEF1A1 or MARK3 as markers to mark the presence of tumor cells in mammals, in man in particular, and possibly their sensitivity to chemotherapies, we also found that inhibition of the activity or expression of eEF1A1 or MARK3 can enable the inhibited growth of tumor cells.

We therefore also provide methods to inhibit the growth of tumor cells, characterized in that the expression or activity of the eEF1A1 or MARK3 protein is inhibited by means of an anti-eEF1A1 or anti-MARK3 inhibitor, respectively.

According to one particular embodiment, the anti-MARK3 inhibitor specifically inhibits the variant of MARK3 whose protein sequence is given under SEQ ID NO: 5. By specific inhibition, is meant that it inhibits the variant concerned without substantially inhibiting the other variants of MARK3.

The anti-eEF1A1 or anti-MARK3 inhibitors inhibiting the activity of eEF1A1 or MARK3, act at the eEF1A1 protein or MARK3 protein, respectively, preventing or limiting its capacity to carry out its biological function. It may, for example, be an antibody whose antigen is an eEF1A1 protein or MARK3 protein, respectively, or at least one epitope of one of these proteins such as defined above and below.

The anti-eEF1A1 inhibitors inhibiting the expression of eEF1A1 or the anti-MARK3 inhibitors inhibiting the expression of MARK3 both act at the transcription of the gene, respectively, encoding eEF1A1 or MARK3, or at the translation of RNA to the protein. In this second case, it may be an interfering RNA which hybridizes with the messenger RNA (mRNA), an expression product of the gene comprising the sequence encoding the eEF1A1 protein or MARK3 protein, respectively, to inhibit translation, either by mere steric hindrance or to promote cleavage of the mRNA.

Interfering RNA technologies and their use in vitro and in vivo are well known to those skilled in the art, and are described in numerous scientific articles and other patent applications.

Depending on the interfering RNA sequences chosen by those skilled in the art, different levels of inhibition can be obtained making it possible to modulate the desired inhibitor effect. Preferably, the interfering RNAs are prepared and chosen to obtain at least 50% inhibition of the expression of the target gene in a cell, even at least 75%, 90%, 95% inhibition, even more than 99% inhibition.

Small Interfering RNAs (siRNAs) are short sequences of around 15 to 30 base pairs (bp) preferably 19 to 25 bp. They comprise a first strand and a complementary strand identical to the RNA targeted region of the RNA of the target gene.

The design and preparation of siRNAs and their use for in vivo and in vitro cell transfection are well known and widely described in numerous publications such as:

U.S. Pat. No. 6,506,559, US2003/0056235, WO99/32619, WO01/75164, WO02/44321, US2002/0086356, WO00/44895, WO02/055692, WO02/055693, WO03/033700, WO03/035082, WO03/035083, WO03/035868, WO03/035869, WO03/035870, WO03/035876, WO01/688836, US2002/0162126, WO03/020931, WO03/008573, WO01/70949, WO99/49029, U.S. Pat. No. 6,573,099, WO2005/00320, WO2004/035615, WO2004/019973, WO2004/015107;

http://www.atugen.com/sirnatechnology.htm,

http://www.alnylam.com/science-technology/index.asp,

http://www.protocl-online.org/prot/ResearchTools/OnlineTools/SiRNADesign/,

http://www.hgmp.mrc.ac.uk./Software/EMBOSS/Apps/sima.html,

http://www.rockefeller.edu/labheads/tuschl/sirna.html,

http://www.upstate.com/browse/categories/siRNA.q.

The siRNAs can be designed and prepared using suitable software available online, for example:
“siSearch Program” http://sonnhammer.cgb.ki.se/siSearch/siSearch1.6html (Improved and automated prediction of effective siRNA”, Chalk A M, Wahlesdelt C and Sonnhammer ELL, Biochemical and Biophysical Research Communications, 2004),
“SiDirect” http://design.maijp/sidirect/index.php (Direct: highly effective, target—specific siRNA design software for mammalian RNA interference, Yuki Naito et al, Nucleic Acids Res., Vol. 32, No. Web Server issue © Oxford University Press 2004),
“siRNA design tool” Whitehead Institute of Biomedical Research, MIT http://jura.wi.mit.edu/pubint/http://iona.wi.mit.edu/siRNACxt/
siRNA wizard, Invitrogen
Error! Hyperlink reference not valid.
“siRNA Target Finder” by Ambion http://www.ambion.com/techlib/misc/siRNAfinder.html
https://www.genscript.com/ssl-bin/app/rnai
http://www.promega.com/siRNADesigner/default.html
http://bioweb.pasteur.fr/seqanal/interfaces/sirna.html
Other programmes are referenced on the site
http://web.mit.edu/mmemanus/www/home1.2files/siRNAs.htm and
http://athena.bioc.uvic.ca/cgi-bin/emboss.pl? action=input& app=sima.

The tools to prepare siRNA and transfect cells are available to the public on simple request on line, e.g., the siRNA vectors marketed by Invitrogen (http://www.invivogen.com/cat.php?ID=3).

Advantageously, the anti-eEF1A1 inhibitor or the anti-MARK3 inhibitor is an RNA interfering which inhibits in vitro and/or in vivo the expression of a gene encoding an eEF1A1 protein or respectively encoding a MARK3 protein. This iRNA is preferably chosen from among antisense RNA and double strand RNA (dsRNA), more preferably a siRNA.

The interfering RNAs are preferably designed to inhibit at least 50%, 75%, 90% or 95% even more than 99% of the expression of an eEF1A1 protein or respectively a MARK3 protein in the cells.

According to one preferred embodiment, the siRNA comprises the following sequence capable of inhibiting the expression of a gene encoding an eEF1A1 protein:

Sense sequence:
5′UGG UGA CAA CAU GCU GGA G 3′
Antisense sequence):
5′ CUC CAG CAU GUU GUC ACC A 3′.

According to one preferred embodiment, the siRNA comprises the following sequence capable of inhibiting the expression of a gene coding for a MARK3 protein:

Sense sequence:
5′ ACA GCA CUA UUC CUG AUC A 3′
Antisense sequence:
5′ UGA UCA GGA AUA GUG CUG U 3′.

According to another preferred embodiment, the siRNA comprises the following sequence capable of inhibiting the expression of a gene coding for the specific variant of the MARK3 protein (SEQ ID NO 5):

Sense sequence:
5′ CCU-CCA-AUA-GAC-AGU-GAA-G 3′
Antisense sequence:
5′ CUU-CAC-UGU-CUA-UUG-GAG-G 3′.

We also provide vectors for the expression of an interfering RNA defined above and below, the vector comprising a sequence coding for the interfering RNA under the control of regulatory elements allowing the expression of the interfering RNA in a host cell. The vectors are known to those skilled in the art and are available, such as the vectors Ambions pSilencer™ 5.1 Retro System (http://www.ambion.com/catalog/CatNun.php?5782) or BLOCK-iT™ Lentiviral RNAi Expression System marketed by Invitrogen (https://catalog.invitrogen.com/index.cfi?fuseaction=viewCatalog.viewProductDetails&productDescription=5549&CMP=LEC-GCMSSEARCH&HQS=block).

We also provide vectors to deliver an interfering RNA into a host cell, characterized in that it comprises an interfering RNA, defined above and below, and means allowing the delivery of the interfering RNA into a host cell. The means are known to those skilled in the art, such as lipid lipofectamines for example (http://www.invitrogen.com/content.cfm?pageid=4005) or Mirus (http://www.mirusbio.com/prducts/mai/index.asp).

We also provide interfering RNA, a vector for its expression or a vector for its delivery such as defined above and below, for their therapeutic use.

We also provide pharmaceutical compositions comprising an antibody, or an interfering RNA according to the invention, or a vector for the expression of the interfering RNA, or a vector for the delivery of an interfering RNA, such as defined above and below, in a pharmaceutically acceptable vehicle. The methods to administer interfering RNA are known to those skilled in the art, the vehicles used depending on the chosen administering route. For example IV injection of a chemically modified siRNA has proved to be effective for the inactivation of genes in vivo (Soutschek J, Akine A, Bramlage B, Charisse K, Constien R, Donoghue M, Elbashir S, Geick A, Hadwiger P, Harborth J, John M, Kesavan V, Lavine G, Pandey R K, Racie T, Rajeev K G, Rohl I, Toudjarska I, Wang G, Wuschko S, Bumcrot D, Koteliansky V, Limmer S, Manoharan M, Vornlocher H P, Therapeutic silencing of an endogenous gene by systemic administration of modified siRNAs, Nature, 2004, 432:173-8).

We also provide for the use of an antibody, or an interfering RNA, or a vector for the expression of the interfering RNA, or a vector to deliver an interfering RNA, such as defined above and below, for the treatment of cancers, more particularly glioblastomas, or for the preparation of a medicinal product intended to treat the diseases.

Persons skilled in the art will know how to choose suitable dosages in relation to the patient and the stage of development of the disease to be treated. This knowledge of patient condition and stage of development of the disease can advantageously be obtained using the diagnosis method.

It is understood that this use can be made in combination with other suitable therapeutic means to treat cancer such as other medicinal products, cytotoxic molecules, antibodies or ligands which can be used in oncology, but also radiation treatment means in particular ionizing radiation or even surgery.

In this case, the antibodies or interfering RNAs are used in combination with the other one or more means simultaneously, together or separately, or at different times. For time-shifted treatment, the antibodies or interfering RNAs can be used before or after the other therapeutic means.

We also provide a variant of the eEF1A1 protein comprising the protein sequence of SEQ ID NO: 1 and a nucleic acid sequence coding for the variant. It also relates an expression vector of a variant of the eEF1A1 protein comprising the nucleic acid sequence under the control of regulatory elements needed for the expression of the protein in a host body. It also relates a host body comprising the expression vector, and a method to prepare the variant of the eEF1A1 protein, characterized in that it comprises steps to culture the host body in a suitable culture medium, then a step to collect the variant of the eEF1A1 protein so produced, and optionally its purification.

We also provide a variant of the MARK3 protein comprising the protein sequence of SEQ ID NO: 5 and a nucleic acid sequence encoding the variant. It also relates an expression vector of a variant of the MARK3 protein comprising the nucleic acid sequence under the control of regulatory elements needed for the expression of the protein in a host body. It also relates a host body comprising the expression vector, and a method to prepare the variant of the MARK3 protein, characterized in that it comprises the steps of to culture the host body in a suitable culture medium, then of collecting the variant of the MARK3 protein thus produced, and optionally its purification.

The methods to clone and express a protein in a host body are well known to those skilled in the art, the regulatory elements which form the vector being selected by such persons according to the chosen host body, but also according to culture conditions and the objective for production of this variant of the eEF1 A1 protein or MARK3 protein, respectively.

One of these objectives can be the preparation and application of a method to screen inhibitors of the expression of an eEF1A1 protein or MARK3 protein, respectively, such as defined previously or in the examples, or inhibitors of the activity of an eEF1A1 protein or MARK3 protein, respectively, the method consisting of contacting at least one candidate inhibitor compound with suitable screening means to allow evidencing of the activity of the compound with respect to the expression of eEF1A1 or MARK3, respectively, or their respective activity, the existence or absence of inhibition.

The screening means are well known to those skilled in the art, such as a host body expressing a reporter gene under the control of a promoter of the eEF1A1 or MARK3 protein respectively, or a host body expressing the eEF1A1 or MARK3 protein, respectively, whose expression level or transcription is controlled by suitable methods, or a host body expressing the eEF1A1 or MARK3 protein, or a suitable reaction medium containing the eEF1A1 or MARK3 protein, respectively, allowing control over the activity of the protein under the effect of the candidate compound(s).

According to one particular embodiment, several candidate compounds are tested, together or separately, the compounds possibly forming a library of compounds to be tested. Advantageously, the compounds are chemical compounds called “small molecules.”

According to one particular embodiment, the selected compounds specifically inhibit the expression or activity of the MARK3 variant whose protein sequence is given under SEQ ID NO: 5.

The exemplary embodiments below better illustrate but do not limit the scope of the disclosure.

### List of Examples:

1. Evidencing of discriminating immunoreactive characteristics in biological fluids of patient populations with tumors versus healthy individuals.

2. Characterization by sequencing and mass spectrometry of the antigen responsible for discriminating immunoreactions.

3. Identity of the antigen protein.

4. Western-blot analysis of antigen-antibody reactions and validation of clinical interest.

5. Impact of antibodies on the viability of tumor cells in vitro.

6. Blocking the proliferation of tumor cells in vitro using siRNAs directed against eEF1A.

7. Evidencing of discriminating immunoreactive characteristics in the biological fluids of patient populations with tumors versus healthy individuals.

8. Characterization by sequencing and mass spectrometry of the antigen responsible for discriminating immunoreactions.

9. Identity of the antigen protein.

10. Western-blot analysis of antigen-antibody reactions and validation of clinical interest.

11. Impact of antibodies on the viability of tumor cells in vitro.

12. Blocking the proliferation of tumor cells in vitro by siRNAs.

13. Blocking the proliferation of tumor cells in vitro by siRNAs directed against MARK3 and specifically against the variant in SEQ ID NO: 5 (clone 2C10).

### Example 1

### Evidencing Discriminating Immunoreactive Characteristics in the Biological Fluids of Patient Populations Suffering from Tumors Versus Healthy Individuals

In this example, cystic fluids taken from solid CNS tumors in various patients were analyzed to identify whether these fluids contain antibodies capable of recognizing proteins expressed by the tumors. For this purpose, the reactivity of the cystic fluids to protein extracts of tumors was tested by Western-blot. The Western-blot in FIG. 1 shows several colored bands evidencing numerous reactivities of modest or very strong intensity vis-à-vis various tumor proteins. For example, strong reactions are noted against proteins with molecular weights estimated at around 36, 41 and 53 kDa. Having regard to the protein fractions used in this Western-blot, it can be concluded that the tumor antigens, against which the antibodies present in the cystic fluids are directed, are located either in the membranes of the tumor cells or in their cytoplasm.

After incubation of the Western-blot imprints with the cystic fluid, the antibodies which interacted with the antigen proteins are detected by conventional methods using secondary antibodies coupled to peroxidase.

The evidencing of these immunoreactions and of the antibodies directed against tumor proteins was continued following the strategy described below. The analysis consisted of identifying the presence of antibodies in the cystic fluids and sera of patients, responsible for immunity responses to various human proteins. Immunoreaction tests of sera and cystic fluids against a bank of expression bacterial clones expressing various proteins of the human repertory allowed a clone to be isolated named according to the nomenclature of the clone bank of ours: clone 1G5. The tested biological fluids (sera of healthy individuals) show very strong reactivity against the clone. Conversely, the cystic fluids of tumors and sera from patients with tumors show much weaker reactivity.

This experiment therefore evidences the existence of antibodies directed against a particular human protein in the biological fluids of healthy individuals or patients with tumors, and the fact that the levels of these antibodies are different depending on the groups of individuals tested. The measurement of these antibody levels is therefore of diagnostic interest to allow easy discrimination between individuals suffering from tumors and perfectly healthy individuals.

### Example 2

### Characterization by Sequencing and Mass Spectrometry of the Antigen Responsible for Discriminating Immunoreactions

The protein which is expressed by the 1G5 was identified. This identification was performed in two different, complementary manners.

First, characterization was based on analysis of the sequence of the cDNA cloned in the expression vector of the clone; secondly, the human polypeptide expressed by this clone was purified and analyzed after trypsin proteolysis using a nanoLC-MS/MS approach (liquid nanochromatography coupled to tandem mass spectrometry analysis) (following the protocol used by Bourges et al, 2004).

The first approach consisted of extracting the plasmid from the bacterial clone derived from the bank stock and placing it in culture. Extraction was made following conventional methods to purify bacterial plasmids (namely and in brief by alkaline lysis and precipitation of the plasmid DNA). The purified plasmids were then sequenced using the enzymatic chain termination technique with fluorescent dideoxynucleotides. The sequence was decrypted by capillary migration on an ABI 3700 sequencer. The sequences were made in the sense and antisense directions and validated by several runs. The use of software such as Autoassembler (Applied Biosystems) allowed the integral consensus sequence to be generated of the cDNA fragment cloned in the plasmid. The sequence was examined in detail to recognize the regions of this sequence which encode the expressed human protein (so-called “coding” sequence).

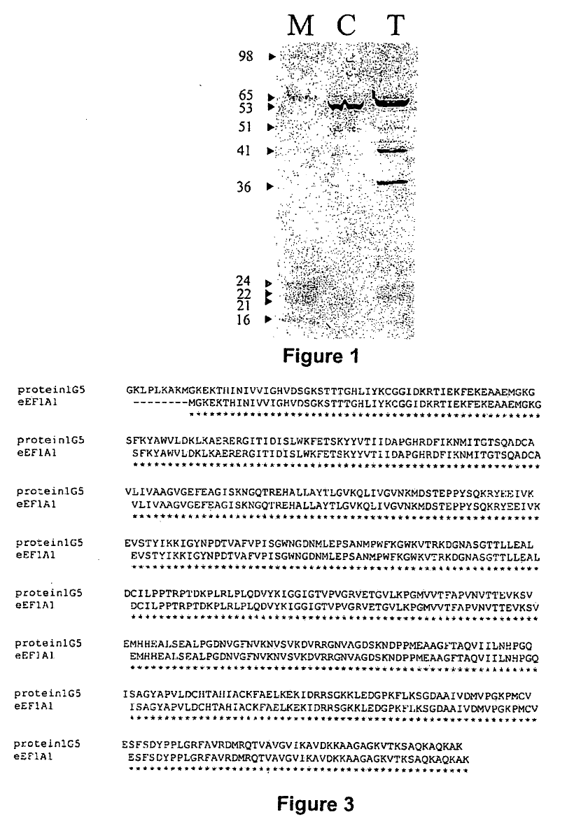

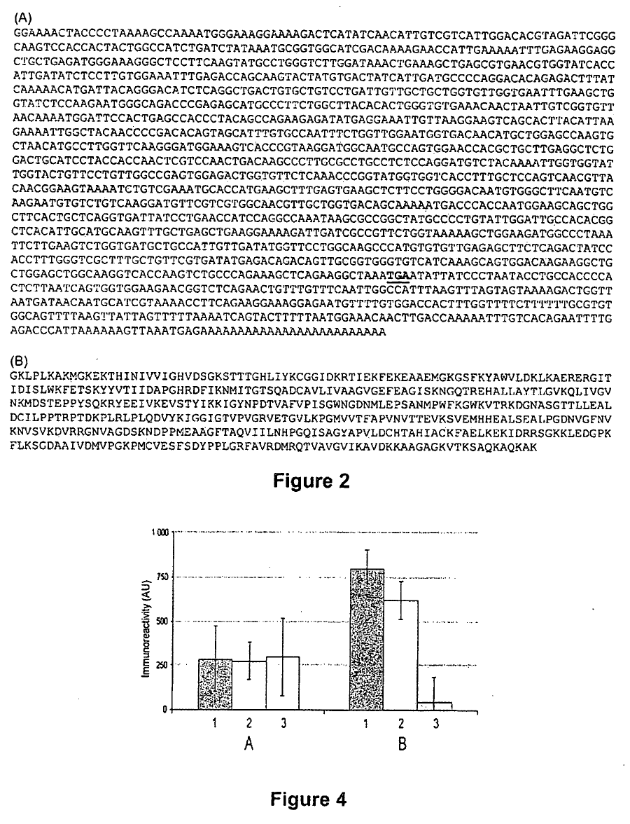

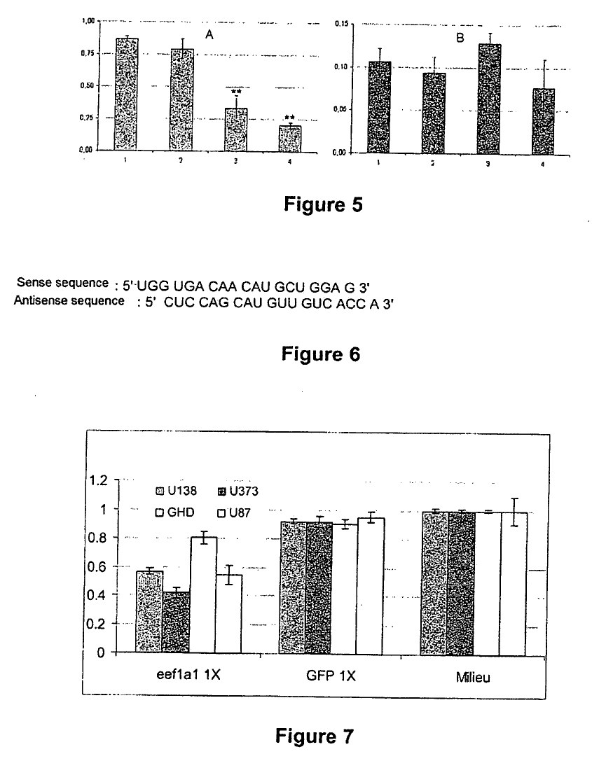

The cDNA sequence coding for the 1G5 clone is shown in FIG. 2. FIG. 2 also shows the sequence of the human protein expressed by the clone such as predicted from the cDNA sequence cloned in the expression vector. The sequence of the plasmid lying upstream of the cDNA and comprising the start codon for synthesis of the protein fragment coded by the cDNA is not shown. The translation stop codon is shown in bold and underlined.

The sequence of the protein expressed by the expression plasmid 1G5 is shown (at B). The conventional one-letter code for the amino acids is used.

The purification of the human protein expressed by the clone was carried out. The experiments were conducted on the basis of standard protocols for purifications and protein manipulation. The bacterial clone 1G5 was therefore placed in culture individually, the bacterial cells were then harvested and lysed in the presence of guanidinium salts. The human proteins were purified having recourse to affinity chromatography using the interactions of the polyhistidine sequences with the resin columns packed with immobilized metals. Owing to the plasmid construction, the human proteins are expressed in the clones in the form of chimera which, at their N-terminal end, integrate a short sequence comprising six consecutive histidine residues. This pattern enables almost selective retention of the human proteins expressed in the clones on the chelating nickel resins. After eluting at acid pH the proteins were subjected to polyacrylanide gel electrophoresis. The major protein band was then cut and treated with trypsin. The peptides obtained were separated by reverse phase chromatography coupled with mass spectrometry analysis. This analysis allows information to be obtained on the primary sequence of the generated trypsic peptides. It therefore provides non-ambiguous validation that the proteins encoded by the cDNAs cloned in the expression vectors are synthesized by the clones and form the antigens detected by immunoreaction.

The 1G5 clone indeed expresses the protein whose sequence is shown in FIG. 2.

### Example 3

### Identity of the Antigen Protein

In silico analysis consisting of comparing the cDNA sequence of the 1G5 clone and the corresponding protein it expresses, with banks of reference sequences of nucleic acids (sequences of cDNA clones and sequence of the human genome) and of human proteins was carried out. With this analysis it was possible to specify the identity of the protein which forms one of the specific antigens responsible for the immunoreactions shown in Example 1. The following information could therefore be obtained:
The 1G5 clone expresses the eEF1A1 protein (it also includes, in its N ter part, 8 additional amino acids).
The sequence of the protein expressed by the 1G5 clone is compared with the reference sequence of the eEF1A1 protein in FIG. 3. Sequence identities are symbolized by asterisks.

### Example 4

### Western-Blot Analysis of Antigen-Antibody Reactions and Validation of Clinical Interest

Immuno-detection tests were undertaken to validate two important parameters: first to verify the specificity of the reactivity of the antibodies present in sera against purified human antigen proteins or their fragments; second to evaluate the biological relevance of the antibodies as indicators of diagnostic interest in oncology.

These tests were conducted using the Western-blot method. To this end, the bacterial clone 1G5 was placed in culture, and the expressed human protein was purified on chelating nickel resin as indicated in the preceding example. The purified protein was subjected to electrophoretic migration on polyacrylamide gel. After electro-transfer onto PVDF membranes, the antigen was detected by impregnating the membranes with varied sera obtained from blood samples of numerous individuals. The cohort of individuals formed for the analysis consisted of 60 healthy individuals; 20 individuals with glial tumors not responding to chemotherapy (treatment with Temodal® or Schering Plough temozolomide); and 14 individuals with glial tumors characterized by objectivised sensitivity to this chemotherapy (on the basis of tumor size regression observed on imaging at three-month interval).

Western-blot analysis allowed the following conclusions to be drawn.

For the 1G5 clone, the sera react against two Western-blot regions, one region corresponding to an antigen having an apparent size of 50 kDA, and one corresponding to a size of 12 kDa. Analysis by enzymatic digestion of the equivalent electrophoresis gel areas followed by nanoLC-MS/MS characterization shows that the region of 50 kDA corresponds to the eEF1A1 protein expressed by the bacterial clone 1G5, and the region of 12 kDA corresponds to a fragmentation peptide generated during purification of the protein. This fragment encompasses the N-terminal part of the antigen produced by the 1G5 clone.

The intensities of the immunodetection reactions obtained on Western-blots were quantified. Analysis of the data obtained individually, on sera of the 84 individuals in the cohort, showed that the response to the peptide of 12 kDA is high in the sera of healthy individuals, and that this response is almost just as intense in the sera of patients with glial tumors characterized by sensitivity to chemotherapy. On the other hand, the reactivity of the sera of patients suffering from glial tumors not responding to chemotherapy was considerably reduced.

This example clearly shows that the detection of serum antibodies directed against the eEF1A1 protein is of diagnostic and prognostic value for the clinical management of patients suffering from glial tumors. The presence or absence of serum antibodies directed against the variant of the eEF1A1 protein (or its fragments) allows the prognostic determination of whether the tumor is likely to be resistant to chemotherapy.

The results given here show that particular epitopes present in the described protein (eEF1A1) can be used to evaluate the variations in antibody levels in the biological fluids of an individual; an evaluation having major clinical interest. Any other biological or artificial, natural or chimerical component which carries the epitopes recognized by the antibodies which are detected in the described method, and are of clinical interest, can be used advantageously to conduct assays within the spirit of the method described in this patent.

FIG. 4 shows Western-blot analysis of the antigen proteins produced by the 1G5 clone. The proteins and peptides resulting from spontaneous fragmentations were subjected to electrophoretic migration on polyacrylamide gel. The immunoreactions were developed conventionally using the Western-blot approach, after impregnating the blots with mixtures of sera derived from various groups of individuals.

The groups of individuals consisted of:
Group 1: normal individuals, controls;
Group 2: patients with gliomas responding positively to chemotherapy;
Group 3: with gliomas not responding to chemotherapy.

The immunoreactivity histograms correspond to the signals recorded in the Western-blots with the following antigens:
A: total protein expressed by the 1G5 clone (corresponding to the eEF1A1 sequence described in Example 3, FIG. 3);
B: 12 kDa fragmentation product of eEF1A1 described in Example 3, FIG. 3.

### Example 5

### Impact of Antibodies on the Viability of Tumor Cells In Vitro

To confirm the predictable therapeutic use of the antibodies directed against the identified tumor antigens, an evaluation was made of the cytotoxic impact of the immunoglobulins extracted from biological fluids on culture tumor cells.

Experimenting consisted of preparing samples of purified immunoglobulins, taken from the sera of healthy individuals and from cystic fluids of individuals with a glial tumor. The immunoglobulins were purified by “Hitrap protein-G” column chromatography, distributed by Amersham (General Electric). The conditions of use conformed strictly to those described by the supplier. The purified immunoglobulins were re-suspended to a titer of 1 mg/ml. Culture tumor cells in vitro were contacted with the purified immunoglobulins at a final concentration of 1 microgram of immunoglobulins per ml in a medium not containing any foetal calf serum. Incubation was continued for 7 days. After this incubation period, the viability of the cells was measured using a conventional cell viability test. The survival rates were calculated against controls corresponding to cell cultures incubated with the preparation and dilution buffers for the immunoglobulins but devoid of immunoglobulins (control buffer).

The immunoglobulins were prepared from cystic fluids taken from tumors of type: oligodendroglioma or meningioma or grade III astrocytoma.

Cell types were represented by primary glioblastomas taken from different patients (2 different glioblastomas), an IMR32 neuroblastoma line, an EJ bladder carcinoma line.

As shown FIG. 5, the purified immunoglobulins of the sera taken from healthy individuals only show very little cytotoxicity with respect to the different cell types. On the other hand, the purified immunoglobulins of cystic fluids of different origins very significantly reduce the survival of glial tumor cells. The survival rate is between 15 and 40% depending on the extracts used. The cells of glioblastoma-type tumors are the most sensitive; the cells of bladder carcinoma are more modestly affected (65 to 75% survival); the viability of neuroblastoma cells is not substantially modified. This latter information proves that at a concentration of 1 microgram of immunoglobulins per ml of medium, the purified immunoglobulins of cystic fluids do not show any non-specific toxicity towards culture tumor cells.

The example leads to the conclusion that the immmunoglobulins which are present in cystic fluids and which react with tumoral antigens have manifest cytotoxic capacity towards tumor cells. These antibodies differ very clearly from the immunoglobulins present in the sera of healthy individuals which do not show any marked anti-tumoral activity. The cytotoxic capacity of the purified immuno globulins of cystic fluids is shown not only with respect to CNS tumor cells but also, although to a lesser extent, with respect to bladder cancer cells, as indicated here. It would appear that generalized use of these antibodies in therapeutic approaches can therefore be taken into consideration for the treatment of CNS tumors and certain forms of other cancers affecting body organs other than the CNS.

FIG. 5 shows the measurement of the cytotoxicity of purified immunoglobulins of cystic fluids or sera vis-à-vis culture tumor cells. The histograms illustrate the survival rates (expressed in arbitrary values) of culture tumor cells after 7 days' incubation in the presence of: 1) control buffer; 2) serum immunoglobulins from healthy individuals; 3) cystic fluid immunoglobulins of oligodendroglial tumor; 4) cystic fluid immunoglobulins from a grade III astocyte tumor. The culture cells are: at A) glioblastomas; at B) neuroblastomas.

The concentrations of immunoglobulins in contact with the cells are set at 1 microgram of immunoglobulin per ml of medium for all tests.

### Example 6

### Blocking the Proliferation of In Vitro Tumor Cells by siRNAs

The siRNA sequence allowing disturbed expression of eEF1A1 proteins was chosen in relation to the cDNA sequence of the 1G5 clone (cf. FIG. 2).

In brief, two sequences were chosen to create a siRNA: namely the sense sequence which has a length of 19 bases homologous to part of the sequence of the messenger RNA encoding the protein; and a sequence that is perfectly complementary to the chosen “sense” sequence. The two sequences have a length of 19 bases. The sense and antisense sequences are given in FIG. 6. The RNA fragments corresponding to the sense sequences and to the complementary sequences were synthesized by chemical route. Double-strand RNAs were then created in vitro by hybridization between the RNAs corresponding to the sense sequences and the fragments corresponding to the complementary sequences. These double-strand RNA fragments were used to transfect culture tumor cells. The transfecting agent here was oligofectamin. Suitable controls were also carried out, in particular: transfection of cells following a strictly identical protocol but not including the siRNAs. The cells used were glioblastoma cells (U373 line having strong in vitro proliferation).

The culture U373 glioblastoma cells were transfected by the siRNAs whose sequences are given FIG. 6. After being kept for 5 days in a growth medium, the cells were counted and the proliferation rate, calculated relative to initial seeding of the medium, was measured. Compared with normal development of the cells under control conditions (proliferation rate set at 100%), the double-strand siRNA directed against eEF1A1 significantly slows down the proliferation of glioblastoma cells. In the presence of siRNA directed against eEF1A1, cell proliferation is only 41% compared with controls.

The transfection tests were conducted on 3 new cell lines: U138, GHD and U87. The results given FIG. 7 confirm the cytotoxic effect of the siRNAs on these glioblastoma cell lines.

“Medium” status corresponds to the control without transfection. “GFP” corresponds to transfection with a siRNA directed against the GFP protein (controls for transfection innocuousness).

### Example 7

### Evidencing Discriminating Immunoreactive Characteristics in the Biological Fluids of Patient Populations Suffering from Tumors Versus Healthy Individuals

In this example, cystic fluids taken from solid CNS tumors in various patients were analyzed to detect whether these fluids contain antibodies capable of recognizing proteins expressed by the tumors. For this purpose, the reactivity of the cystic fluids vis-à-vis protein extracts of tumors was tested by Western-blot. The Western-blot illustrated in FIG. 8 shows several colored bands evidencing numerous reactivities of modest or very strong intensity vis-Avis various tumor proteins. For example strong reactions are noted against proteins with molecular weights estimated at around 36, 41 and 53 kDa. Having regard to the protein fractions used in this Western-blot, it can be concluded that the tumor antigens, against which the antibodies present in the cystic fluids are directed, are located either in the membranes of the tumor cells or in their cytoplasm.

After incubation of the Western-blot imprints with the cystic fluid, the antibodies which interacted with the antigen proteins were detected by conventional methods using secondary antibodies coupled to peroxidise.

The evidencing of these immunoreactions and antibodies directed against tumor proteins was continued following the strategy described below. The analysis consisted of revealing the presence of antibodies, in the cystic fluids and also in the sera of patients, responsible for immunity responses to various human proteins. Immunoreaction tests of the sera and cystic fluids against a bank of bacterial expression clones expressing various proteins of the human repertoire, allowed the isolation of a clone named in accordance with the nomenclature of the clone bank particular to us: clone 2C10. The tested biological fluids (tumor cystic fluids and sera from patients with tumors) showed very strong reactivity towards the clone. Conversely, the sera of healthy individuals showed much weaker reactivity, even the near-absence of reactivity.

This experiment therefore evidences the existence of antibodies directed against a particular human protein in the biological fluids of healthy individuals or patients suffering from tumors, and the fact that the levels of these antibodies are different depending on the groups of individuals tested. The measurement of these antibody levels therefore has the diagnostic advantage of allowing easy discrimination between individuals suffering from tumors and perfectly healthy individuals.

### Example 8

### Characterization by Sequencing and Mass Spectrometry of the Antigen Responsible for Discriminating Immunoreactions

The protein which is expressed by the 2C10 clone was identified. This identification was achieved in two different, complementary manners.

First, characterization was based on the analysis of the cDNA sequence cloned in the expression vector of the clone; second, the human polypeptide expressed by this clone was purified and analyzed, after trypsin proteolysis, using a nanoLC-MS/MS approach (liquid nanochromatography coupled with analysis by tandem mass spectrometry) (following the protocol used by Bourges et al, 2004).

The first approach consisted of extracting the plasmid from the bacterial clone derived from the bank stock and placed in culture. Extraction was made using conventional purification methods for bacterial plasmids (namely and in brief, by alkaline lysis and precipitation of the plasmid DNA). The purified plasmids were then sequenced using an enzymatic chain termination technique with fluorescent dideoxynucleotides. The sequence was decrypted by capillary migration on ABI 3700 apparatus. The sequences were conducted in the sense and antisense directions and validated by several runs. The use of software such as Autoassembler (Applied Biosystems) allowed generation of the integral consensus sequence of the cDNA fragment cloned in the plasmid. The sequence was examined in detail to recognize the regions of this sequence which encode the expressed human protein (so-called “coding” sequence).

The cDNA sequence coding for the 2C10 clone is shown FIG. 9. FIG. 9 also shows the sequence of the human protein expressed by the clone such as predicted from the cDNA sequence cloned in the expression vector. The sequence of the plasmid lying upstream of the cDNA and comprising the start codon for synthesis of the protein fragment encoded by the cDNA is not shown. The translation stop codon is shown in bold and underlined.

The sequence of the protein expressed by the expression plasmid 2C10 is shown (at B). The conventional one-letter code for amino acids is used. For the 2C10 clone, the sequence of amino acids specific to the variant identified is underlined.

Purification of the human protein expressed by the clone was carried out. The experiments were conducted on the basis of standard purification and protein manipulation protocols. The bacterial clone 2C10 was therefore placed in culture individually, the bacterial cells were then harvested and lysed in the presence of guanidinium salts. The human proteins were purified having recourse to affinity chromatography using the interactions of the polyhistidine sequences with resin columns loaded with immobilized metals. Owing to the plasmid construction, the human proteins are expressed in the clones in the form of chimera which, at their N-terminal end, integrate a short sequence comprising six consecutive histidine residues. This pattern allows near-selective retention of the human proteins expressed on the clones on the nickel chelating resins. After eluting at acid pH, the proteins were subjected to electrophoresis on polyacrylamide gel. Then the major protein band was cut and treated with trypsin. The peptides obtained were separated by reverse phase chromatography coupled with mass spectrometry analysis. This analysis allows information to be obtained on the primary sequence of the trypsic peptides generated. It therefore enables non-ambiguous validation that the proteins encoded by the cDNAs cloned in the expression vectors are synthesized by the clones and form the antigens detected by immunoreaction.

The 2C10 clone indeed expresses the protein whose sequence is given in FIG. 9.

### Example 9

### Identity of the Antigen Protein

In silico analysis was carried out consisting of comparing the cDNA sequence of clone 2C10, and the corresponding protein it expresses, against banks of reference nucleic acid sequences (sequences of cDNA clones and sequence of the human genome) and of human proteins. With this analysis it was possible to specify the identity of the protein which forms one of the specific antigens responsible for the immunoreactions shown in Example 7. The following information was therefore obtained:
The 2C10 clone expresses an unknown variant of a fragment of the MARK3 protein. The MARK3 protein is a protein kinase. The MARK3 gene is located at 14q32.3. The sequence of the MARK3 fragment expressed by the 2C10 clone, which corresponds to the antigen responsible for the immunoreactivities of the biological fluids, has 84% identity with the known reference sequence of an isoform of MARK 3 (isoform 4 of MARK3) carrying the nomenclature P27448-4 in the Swiss-Prot base). It consists of the 279 C terminal amino acids but includes an additional sequence of 52 amino acids, and is located 215 amino acids upstream from the C terminal amino acid of the reference sequence (cf. sequence underlined in FIG. 9). If abstraction is made of this additional sequence of 52 amino acids, the sequence of the protein expressed by the 2C10 clone has 100% identity with the C ter end of isoform 4 of MARK3. The protein sequence encoded by this clone is original and is not referenced in Genbank (http://www.ncbi.nlm.nih.gov/). The sequence of the cDNA fragment present in clone 2C10 was compared with the genomic sequence of the gene responsible for the expression of MARK3 in human tissues. This analysis revealed that the sequence of 52 original amino acids, present in the variant of MARK3 described here, is coded by a cryptic exon of 156 bases which was conserved during splicing of the normal transcript of MARK3.

The sequence of the protein expressed by the 2C10 clone was compared with the reference sequence of the MARK3 protein in FIG. 10. The sequence identities are symbolized by asterisks.

### Example 10

### Western-Bot Analysis of Antigen-Antibody Reactions and Validation of Clinical Interest

Immunodetection tests were undertaken to validate two important parameters: first to verify the specificity of the reactivity of the antibodies present in the sera vis-à-vis purified antigenic human proteins or their fragments; second, to evaluate the biological relevance of the antibodies as indicators of diagnostic interest in oncology.

These tests were conducted using the Western-blot method. For this purpose, the bacterial clone 2C10 was placed in culture, and the expressed human protein was purified on nickel chelating resin as indicated in the preceding example. The purified protein was subjected to polyacrylamide gel electrophoretic migration. After electro-transfer onto PVDF membranes, the antigen was detected by impregnating the membranes with varied sera obtained from blood samples taken from numerous individuals. The cohort of individuals formed for the analysis consisted of 50 healthy individuals; 20 individuals with glial tumors not responding to chemotherapy (treatment with Temodali or Schering Plough temozolomide); and 14 individuals having glial tumors characterized by objectivized sensitivity to this chemotherapy (on the basis of tumor size regression observed on imaging at three-month interval).

Analysis of the Western-blots allowed the following conclusions to be drawn.

For the 2C10 clone, the sera react against two Western-blot regions, one region corresponding to an antigen having an apparent size of 45 kDA, and one corresponding to a size of 11 kDa. Analysis by enzymatic digestion of the equivalent areas of the electrophoresis gel, followed by characterization with nanoLC-MS/MS showed that the 45 kDa region corresponds to the identified original variant of the MARK3 protein expressed by the bacterial clone 2C10, and the area of 11 kDa corresponds to a fragmentation peptide generated during purification of the protein. This fragment encompasses the N-terminal part of the antigen produced by the 2C10 clone.

The intensities of the immune-detection reactions obtained on the Western-blots were quantified. Analysis of the data obtained individually with the sera of the 84 individuals of the cohort showed that the response to the protein of 45 kDa and to the peptide of 11 kDa was near-inexistent in the sera of healthy individuals, and that this response was intense in the sera of patients with glial tumors (with no major distinction between the tumors characterized by sensitivity to chemotherapy and those which are resistant thereto).

This example clearly shows that the detection of serum antibodies directed against the MARK3 protein has a diagnostic and prognostic value for the clinical management of patients suffering from glial tumors. The onset of serum antibodies directed against the variant of the MARK3 protein (or its fragments) obviously teaches the presence of solid tumors of the central nervous system.

The results given here show that particular epitopes present in the described protein (variant of MARK3) allow an evaluation of variations in antibody levels in the biological fluids of an individual; an evaluation of major clinical interest. Any other biological or artificial, natural or chimerical component which comprises the epitopes recognized by the antibodies which are detected in the described method, and of clinical interest, can be used advantageously to conduct assays in the spirit of the method described in this patent.

FIG. 11 shows the Western-blot analysis of the antigenic proteins produced by the 2C10 clone. The proteins and peptides resulting from spontaneous fragmentations were subjected to electrophoretic migration on polyacrylamide gel. The immunoreactions were developed in conventional manner using the Western-blot approach after impregnating the blots with mixtures of sera derived from various groups of individuals.

The groups of individuals consisted of:
Group 1: normal individuals, controls;
Group 2: patients with gliomas responding positively to chemotherapy;
Group 3: patients with gliomas not responding to chemotherapy.

The immunoreactivity histograms correspond to the signals recorded in the Western-blots with the following antigens:
A: total protein expressed by the 2C10 clone (corresponding to the domain of the isoform 4 variant of MARK3 described in Example 9, FIG. 10);
B: 11 kDa fragmentation product of the domain of the isoform variant 4 of MARK3 described in Example 9, FIG. 10.

### Example 11

### Impact of the Antibodies on the Viability of In Vitro Tumor Cells

To confirm the predictable therapeutic use of the antibodies directed against the identified tumoral antigens, an evaluation was made of the cytotoxic impact of the immunoglobulins extracted from biological fluids on culture tumor cells.

Experimenting consisted of preparing samples of purified immunoglobulins taken from the sera of healthy individuals and from cystic fluids of individuals suffering from a glial tumor. The immunoglobulins were purified by “Hitrap protein-G” column chromatography distributed by Amersham (General Electric). The conditions of use conformed strictly to those described by the supplier. The purified immunoglobulins were re-suspended to a titer of 1 mg/ml. Culture tumor cells in vitro were placed in contact with the purified immunoglobulins at a final concentration of 1 microgram of immunoglobulins per ml in a medium not containing any foetal calf serum. Incubation was continued for 7 days. After this incubation period, the viability of the cells was measured using a conventional cell viability test. The survival rates were calculated relative to controls corresponding to the cell cultures placed in incubation with preparation and dilution buffers for immunoglobulins but devoid of immunoglobulins (control buffer).

The immunoglobulins were prepared from cystic fluids taken from tumors of type: oligodendrogliomas, meningiomas or grade III astrocytoma.

The cell types were represented by primary glioblastomas taken from different patients (2 different glioblastomas), an IMR32 neuroblastoma line, an EJ bladder carcinoma line.

As shown in FIG. 12, the purified immunoglobulins of the sera from healthy individuals show only very little cytotoxicity vis-à-vis the different cell types. On the other hand, the purified immunoglobulins from the cystic fluids of different origins reduce the survival of glial tumor cells most significantly. The survival rate is between 15 and 40% depending on the extracts used. The tumor cells of glioblastoma type are the most sensitive; the bladder carcinoma cells are more modestly affected (65 to 75% survival); the viability of neuroblastoma cells is not substantially modified. This latter information proves that, at a concentration of 1 microgram of immunoglobulins per ml of medium, the purified immunoglobulins of the cystic fluids do not show any non-specific toxicity vis-à-vis culture tumor cells.

This example leads to the conclusion that the immunoglobulins which are present in the cystic fluids and which react with tumoral antigens have obvious cytotoxic capacity vis-à-vis tumor cells. These antibodies differ very distinctly from the immunoglobulins present in the sera of healthy individuals which do not show any marked anti-tumor activity. The cytotoxic capacity of the purified immunoglobulins of cystic fluids is shown not only vis-à-vis CNS tumor cells but also, even though to a lesser extent, vis-à-vis bladder cancer cells as indicated here. It would therefore appear that the generalized use of these antibodies in therapeutic approaches can be considered for the treatment of CNS tumors and certain forms of other cancers affecting organs other the CNS.

FIG. 12 shows the measured cytotoxicity of the purified immunoglobulins of cystic fluids or sera on culture tumor cells. The histograms represent survival rates (expressed in arbitrary values) of culture tumor cells after 7 days' incubation in the presence of: 1) control buffer; 2) immunoglobulins of sera from healthy individuals; 3) immunoglobulins of a cystic fluid from an oligodendroglial tumor; 4) immunoglobulins of a cystic fluid from a grade III astocyte tumor. The culture cells are: at A) glioblastomas; at B) neuroblastomas.

The concentrations of immunoglobulins in contact with the cells were set at 1 microgram of immunoglobulins per ml of medium in all tests.

### Example 12

### Blocking the Proliferation of In Vitro Tumor Cells by siRNAs

The siRNA sequence allowing disturbed expression of the MARK3 protein was chosen in relation to the cDNA sequence of the 2C10 clone (cf. FIG. 9).

In brief, two sequences were chosen for the creation of an siRNA; namely the sense sequence which has a length of 19 bases homologous to part of the sequence of the messenger RNA encoding the protein; and a perfectly complementary sequence to the chosen “sense” sequence. The two sequences have a length of 19 bases. The sense and antisense sequences are shown in FIG. 13:

Sense sequence:
5′ ACA GCA CUA UUC CUG AUC A 3′
Antisense sequence:
5′ UGA UCA GGA AUA GUG CUG U 3′.

The RNA fragments corresponding to the sense sequences and to the complementary sequences were synthesized via chemical route. Double-strand RNAs were then created in vitro by hybridization between the RNAs corresponding to the sense sequences and the fragments corresponding to the complementary sequences. These double-strand RNA fragments were used to transfect culture tumor cells. The transfecting agent here was oligofectamin. Appropriate controls were also conducted in particular: transfection of cells following a strictly identical protocol but not including the siRNAs. The cells used were glioblastoma cells (line U373 having strong in vitro proliferation).

The cells of culture U373 glioblastomas were transfected with the siRNAs whose sequences are given in FIG. 13. After being kept for 5 days in growth medium, the cells were counted and the proliferation rate, calculated relative to initial seeding of the medium, was measured. Compared with normal development of the cells under control conditions (proliferation rate set at 100%) the double-strand siRNA directed against MARK3 significantly slowed down the proliferation of glioblastoma cells. In the presence of the siRNA directed against MARK3, cell proliferation was only 74% compared with controls.

### Example 13

### Blocking the Proliferation of In Vitro Tumor Cells by siRNAs Directed Against MARK3 and Specifically Against the Variant of SEQ ID NO: 5 (Clone 2C10).

Two types of tumor cell lines were used: the human glial tumor cell lines (glioblastomas) called U87 and U373. siRNAs are transfected in the culture cells using a conventional method (use of the transfection agent oligofectamin; siRNA concentration of 450 nM). Cell survival was measured after transfection. The status “Medium” corresponds to the control without transfection. “GFP” corresponds to transfection with a siRNA directed against the GFP protein (controls for transfection innocuousness). The statuses 2C10-3 and MARK 3× correspond to the impact of the siRNAs directed against the transcript of the 2C10 antigen and against MARK3.

The so-called “MARK3” sequence targets a region of the transcript that is identical between the different variants of MARK3:

Sense sequence:
5′ UGA-UCA-GGA-AUA-GUG-CUG-U 3′
Antisense sequence:
5′ ACA-GCA-CUA-UUC-CUG-AUC-A 3′.

The status 2C10-3 corresponds to the use of a siRNA which is directed against that part of the original sequence of the transcript which encodes the sequence of 52 amino acids signing the original variant:

Sense sequence:
5′ CCU-CCA-AUA-GAC-AGU-GAA-G 3′
Antisense sequence:
5′ CUU-CA-UGU-CUA-UUG-GAG-G 3′.

The results obtained shown in FIG. 14 indicate, for both types of siRNA, a significant reduction in proliferation compared with the different controls (Medium and GFP).

On the U87 line, the 2C10-3 siRNA shows good cytotoxic activity (as shown in the histogram transmitted last Tuesday) which is greater than that obtained with the MARK3 siRNA.

### BIBLIOGRAPHICAL REFERENCES

The subject matter of the following bibliographical references is incorporated herein by reference.
Beghini A, Magnani I, Roversi G, Piepoli T, Di Terlizzi S, Moroni R F, Polio B, Fuhrman Conti A M, Cowell J K, Finocchiaro G, Larizza L. Oncogene. 2003, 22, 2581-91.
Blomberg K, Hurskainen P, Hemmila I. Clin Chem. 1999, 45, 855-61.
Booth J C, Hannington G, Bakir T M, Stern H, Kangro H, Griffiths P D, Heath R B. J Clin Pathol. 1982, 35, 1345-8.
Bourges I, Ramus C, Mousson de Camaret B, Beugnot. R, Remacle C, Cardol P, Hofhaus G, Issartel J P. Biochem. J. 2004, 383, 491-9.
Bruner J M, Inouye L, Fuller G N, Langford L A. Cancer. 1997, 79, 796-803.
Burnette W N., Anal Biochem. 1981, 112, 195-203.
Cairncross J G, Ueki K, Zlatescu M C, Lisle D K, Finkelstein D M, Hammond R R, Silver J S, Stark P C, Macdonald D R, Ino Y, Ramsay D A, Louis D N. J Natl Cancer Inst. 1998, 90, 1473-9.
Chatel M, Brucher J-M. dans “Neuro-oncologie”, Editeur J. Hildebrand, Doin 2001; p 25-34.
Coons S W, Johnson P C, Scheithauer B W, Yates A J, Pearl D K. Cancer. 1997, 79, 1381-93.
Ditzel H J, Masaki Y, Nielsen H, Farnaes L, Burton D R. Proc Natl Acad Sci USA. 2000, 97, 9234-9.
Daumas-Duport C, Figarella-Branger D., in “Classification histopronostiques des tumeurs cérébrales gliales et leurs impacts en thérapeutique” Editions espaces 34, 2002; p 11-48.
Ejiri S I. Biosci. Biotechnol. Biochem. 2002, 66, 1-21.
Elbashir S M, Harborth J, Lendeckel W, Yalcin A, Weber K, Tuschl T. Nature. 2001, 411, 494-8.
Ellington A D, Szostak J W. Nature 1990, 346, 818-22.
Engvall E, Perlmanr P. Immunochemistry. 1971, 8, 871-874.
Engvall E, Jonsson K, Perlmann P. Biochim Biophys Acta. 1971, 251, 427-34.
Engvall E, Perlmann P. J. Immunol. 1972, 109, 129-35.
Enomoto K, Aono Y, Mitsugi T, Takahashi K, Suzuki R, Preaudat M, Mathis G, Kominami G, Takemoto H. J Biomol Screen. 2000, 5, 263-8.
Fagerstam L G, Frostell A, Karlsson R, Kullman M, Larsson A, Malnqvist M, Butt H. J Mol Recognit. 1990, 3, 208-14.
Gao C, Mao S, Lo C H, Wirsching P, Lerner R A, Janda K D. Proc Natl Acad Sci USA. 1999, 96, 6025-30.
Grant A G, Flomen R M, Tizard M L, Grant D A. Int J. Cancer. 1992, 50, 740-5.
Hechemy K, Stevens R W, Gaafar H A., Appl Microbiol. 1974, 28, 306-11.
Hulett H R, Bonner W A, Barrett J, Herzenberg L A. Science. 1969, 166, 747-9.
Karagiannis T C, El-Osta A. Cancer Gène Ther. 2005 May 13.
Khalyfa A, Carlson B M, Carlson J A, Wang E. Dev Dyn. 1999, 216, 267-73.
Kato T, Satoh S, Okabe H, Kitahara O, Ono K, Kihara C, Tanaka T, Tsunoda T, Yamaoka Y, Nakamura Y, Furukawa Y. Neoplasia. 2001, 3, 49.
Kiernan J A. Histological and histochemical methods. Theory and practise. Third Edition. Oxford: Butterworth-Heinemann 1999.
Knappik A, Ge L, Honegger A, Pack P, Fischer M, Wellnhofer G, Hoess A, Wolle J, Pluckthun A, Vimekas B. J Mol Biol 2000, 296, 57-86.
Kusnezow W, Hoheisel J D. J Mol Recognit. 2003, 16, 165-76.
Laemmli U K. Nature. 1970, 227, 680-5.
Lee J M. Reprod. Biol. Endocrinol. 2003, 1, 69.
Mansilla F, Hansen L L, Jakobsen H, Kjeldgaard N O, Clark B F, Knudsen C R. Biochim Biophys Acta. 2005, 1727, 116-24.
Mathis G. Clin. Chem. 1995, 41, 1391-7.
Merchant M, Weinberger S R. Electrophoresis. 2000, 21, 1164-77.
Mittler M A, Walters B C, Stopa E G. J. Neurosurg. 1996, 85, 1091-4.
Ohkouchi K, Mizutani H, Tanaka M, Takahashi M, Nakashima K, Shimizu M. Int Immunol 1999, 11, 1635-40.
Parks D R, Herzenberg L A. Methods Enzymol. 1984, 108, 197-241.
Peluso P, Wilson D S, Do D, Tran H, Venkatasubbaiah M, Quincy D, Heidecker B, Poindexter K, Tolani N, Phelan M, Witte K, Jung L S, Wagner P, Nock S. Anal Biochem. 2003, 312, 113-24.
Shen R, Su Z Z., Olsson C A, Fisher P B. Proc Natl Acad Sci USA 1995, 92, 6778-82.
Singer J M, Plotz C M, Pader E, Elster S K. Am J Clin Pathol. 1957, 28, 611-7.
Szabo A, Stolz L, Granzow R. Curr Opin Struct Biol. 1995, 5, 699-705.
Szollosi J, Damjanovich S, Matyus L. Cytometry. 1998, 34, 159-79.
Towbin H, Staehelin T, Gordon J. Proc. Natl Acad Sci USA. 1979, 76, 4350-4.
Tuerk C, Gold L. Science 1990, 249, 505-10.
Ueda H, Kubota K, Wang Y, Tsumoto K, Mahoney W, Kumagai I, Nagamune T. Biotechniques. 1999, 27, 738-42.
Vikinge T P, Askendal A, Liedberg B, Lindahl T, Tengvall P. Biosens Bioelectron. 1998, 13, 1257-62.
Weinberger S R, Morris T S, Pawlak M. Pharmacogenomics 2000, 1, 395-416.
Xu Y, Piston D W, Johnson C H. Proc Natl Acad Sci USA 1999, 96, 151-6.
Yalow R S, Berson S A. J Clin Invest. 1960, 39, 1157-75.

1-38. (canceled)

39. A method for detecting presence or absence of a tumor in a mammal and/or its sensitivity to chemotherapies, comprising, on a biological sample from said mammal, detecting and/or quantifying:
presence of an eEF1A1 protein, and/or
presence of antibodies directed against an eEF1 A1 protein or a fragment comprising at least one epitope of the eEF1A1 protein, and/or
presence of a MARK3 protein, and/or
presence of antibodies directed against a MARK3 protein or a fragment comprising at least one epitope of the MARK3 protein.

40. The method according to claim 39, wherein the biological sample is at least one selected from the group consisting of blood serum, lymph, cystic fluid, cerebro-spinal fluid (CSF) and tissue homogenates.

41. The method according to claim 39, further comprising detecting and quantifying at least one other biological marker characteristic of the presence and/or invasiveness of a tumor.

42. The method according to claim 39, wherein the presence of the eEF1A1 protein is detected and/or quantified by antibodies directed against the eEF1A1 protein or at least one epitope of the eER1A1.

43. The method according to claim 42, wherein the antibodies are polyclonal or monoclonal antibodies.

44. The method according to claim 39, wherein the presence of antibodies directed against an eEF1A1 protein or a fragment containing at least one epitope of the eEF1A1 protein is detected and/or quantified by an antigen comprising at least one epitope of an eEF1A1 protein.

45. The method according to claim 39, further comprising comparing results obtained from the detection and/or quantification with a reference value characteristic of the presence of a tumor and/or with a reference value characteristic of the absence of a tumor.

46. The method according to claim 39, wherein the eEF1A1 protein comprises the protein sequence given of SEQ ID NO: 1.

47. The method according to claim 39, wherein the presence of the MARK3 protein is detected and/or quantified by antibodies directed against the MARK3 protein or at least one epitope of the MARK3 protein.

48. The method according to claim 47, wherein the antibodies are polyclonal or monoclonal antibodies.

49. The method according to claim 39, wherein the presence of antibodies directed against a MARK3 protein or a fragment containing at least one epitope of the MARK3 protein is detected and/or quantified by an antigen comprising at least one epitope of a MARK3 protein.

50. The method according to claim 39, further comprising comparing results obtained from the detection and/or quantification with a reference value characteristic of the presence of a tumor and/or with a reference value characteristic of the absence of a tumor.

51. The method according to claim 39, wherein the MARK3 protein is a variant comprising the protein sequence of SEQ ID NO: 5.

52. A diagnostic kit that performs the method according to claim 39, comprising means to detect and/or quantify, on a biological sample:
presence of an eEF1A1 protein, and/or
presence of antibodies directed against an eEF1A1 protein or a fragment containing at least one epitope of the eEF1A1 protein, and/or
presence of a MARK3 protein, and/or
presence of antibodies directed against a MARK3 protein or a fragment containing at least one epitope of the MARK3 protein.

53. Antibodies directed against an eEF1A1 protein or a fragment containing at least one epitope of the eEF1A1 protein that bind specifically to the eEF1A1 protein or to at least one epitope of the eEF1A1 protein.

54. Antibodies directed against a MARK3 protein or a fragment containing at least one epitope of the MARK3 protein that bind specifically to the MARK3 protein or to at least one epitope of the MARK3 protein.

55. The antibodies according to claim 54, that bind specifically to a variant of the MARK3 protein containing the protein sequence according to SEQ ID NO: 5, or to at least one epitope of that protein.

56. A method of inhibiting growth of tumor cells in vitro comprising inhibiting activity of an eEF1A1 protein and/or of a MARK3 protein by an antibody or an interfering RNA which inhibits expression of a gene encoding the eEF1A1 protein and/or the MARK3 protein, respectively.

57. Interfering RNA that inhibits in vitro and/or in vivo expression of a gene encoding an eEF1A1 protein or a gene encoding a MARK3 protein.

58. The interfering RNA according to claim 57, which is selected from the group consisting of antisense RNAs and double-strand RNAs (dsRNA).

59. The interfering RNA according to claim 58, wherein the double strand RNA is a siRNA.

60. The interfering RNA according to claim 59, comprising the following sequence capable of inhibiting the expression of a gene encoding a specific variant of the eEF1A1 protein:
Sense sequence:
5′ UGG UGA CAA CAU GCU GGA G 3′
Antisense sequence:
5′ CUC CAG CAU GUU GUC ACC A 3′.

61. The interfering RNA according to claim 59, comprising the following sequence capable of inhibiting the expression of a gene encoding the MARK3 protein:
Sense sequence:
5′ ACA GCA CUA UUC CUG AUC A 3′
Antisense sequence:
5′ UA UCA GGA AUA GUUG CUG U 3′.

62. The interfering RNA according to claim 57, which specifically inhibits a variant of the MARK3 protein containing the protein sequence of SEQ ID NO: 5.

63. The interfering RNA according to claim 62, comprising a siRNA comprising the following sequence:
Sense sequence:
5′ CCU-CCA-AUA-GAC-AGU-GAA-G 3′
Antisense sequence:
5′ CUU-CAC-UGU-CUA-UUG-GAG-G 3′.

64. A vector that expresses the interfering RNA according to claim 57, comprising a sequence coding for said interfering RNA under control of regulation elements allowing expression of said interfering RNA in a host cell.

65. A vector that delivers an interfering RNA to a host cell, comprising an interfering RNA according to claim 57 and means allowing the delivery of said interfering RNA into said host cell.

66. A pharmaceutical composition comprising an antibody according to claim 53, in a pharmaceutically acceptable vehicle.

67. A method for treating cancer comprising administering an effective amount of an antibody according to claim 53 to a mammal.

68. The method for treating cancer according to claim 67, wherein the cancer is a glioblastomas.

69. A variant of a MARK3 protein comprising the protein sequence of SEQ ID NO: 5.

70. A nucleic acid sequence encoding a variant of the MARK3 protein according to claim 69.

71. A vector of expression of a variant of the MARK3 protein comprising a nucleic acid sequence according to claim 70 under control of regulation elements required for expression of said protein in a host body.

72. A host body comprising an expression vector according to claim 71.

73. The method according to claim 69, comprising culturing a host body according to claim 72 in a suitable culture medium, followed by collecting the variant of the MARK3 protein produced and, optionally, purifying it.

74. A pharmaceutical composition comprising an interfering RNA according to claim 57, in a pharmaceutically acceptable vehicle.

75. A pharmaceutical composition comprising a vector for expression of the interfering RNA according to claim 64, in a pharmaceutically acceptable vehicle.

76. A pharmaceutical composition comprising a vector for delivery of an interfering RNA according to claim 65 in a pharmaceutically acceptable vehicle.

77. A method for treating cancer comprising administering an effective amount of an interfering RNA according to claim 57 to a mammal.
